(19) United States
(12) Patent Application Publication
(10) Pub. No.: US 2024/0358937 A1
(43) Pub. Date: Oct. 31, 2024

(54) INSUFFLATOR AND A METHOD FOR INSUFFLATING A CAVITY IN A HUMAN OR ANIMAL BODY

(71) Applicant: PALLIARE LIMITED, Galway (IE)

(72) Inventor: John O’DEA, Galway (IE)

(73) Assignee: PALLIARE LIMITED, Galway (IE)

(21) Appl. No.: 18/649,032

(22) Filed: Apr. 29, 2024

(30) Foreign Application Priority Data
Apr. 27, 2023 (IE) S2023/0132
Oct. 20, 2023 (IE) S2023/0437

(51) Int. Cl. A61M 13/00

(52) U.S. Cl. CPC A61M 13/003; A61M 2205/3331; A61M 2205/52

(57) ABSTRACT

An insufflator (1) monitors the rate of insufflating of the peritoneal cavity (3) in the body (5) of a human subject by monitoring the increase in cavity pressure per unit time in order to avoid bradycardia (slow heart rate). A plurality of reference values of the maximum increases in cavity pressure per unit time is stored in a look-up table (23) for different body cavities for male and female subjects of different age ranges and different body mass index ranges. The particulars of the cavity (the peritoneal cavity 3) the sex, age and body mass index of the subject are inputted into the insufflator (1) through a touchscreen (30), and a microprocessor (20) determines the appropriate maximum increase in the cavity pressure per unit time from the look-up table (23). If during insufflating of the cavity, the microprocessor (20) determines from signals read from a pressure sensor (22) that the increase in cavity pressure per unit time exceeds the appropriate maximum increase in cavity pressure per unit time determined from the look-up table, the microprocessor (20) operates a flow controller (14) to reduce the rate of delivery of insufflating gas to the cavity (3) until the increase in cavity pressure per unit time falls to or below the appropriate maximums increase in cavity pressure per unit time in order to avoid bradycardia.

### FIELD OF THE INVENTION

The present invention relates to an insufflator, and in particular, to an insufflator for insufflating a cavity in the body of a human or animal subject, and the invention also relates to a method for insufflating a cavity in the body of a human or animal subject.

### BACKGROUND TO THE INVENTION

In minimally invasive procedures, for example, minimally invasive surgical and investigative procedures, for example, laparoscopic procedures, and indeed, endoscopic procedures, the cavity, vessel, lumen or organ within which the procedure is to be carried out is insufflated. In general, the cavity, vessel, lumen or organ is insufflated by a suitable insufflating gas, which in general, is carbon dioxide. While in the past it was believed insufflating a cavity, vessel, lumen or organ in a subject had little or no side effects, it has been found that the rate at which the cavity, vessel, lumen or organ is insufflated is critical. Insufflating a cavity, vessel, lumen or organ at too high a rate can cause bradycardia (a slow heart rate). It has been found that rapid insufflation of the peritoneal cavity can cause a sudden increase in intraperitoneal pressure, which may lead to reflex-mediated cardiac inhibition through the vagus nerve. This is known as the Bezold-Jarisch reflex, and can result in bradycardia, hypotension, and even cardiac arrest. A detailed discussion on the problems which may arise in laparoscopic procedures resulting from insufflating of the cavity, in which the procedure is being carried out, is provided in a paper entitled “Cardiovascular and Ventilatory Consequences of Laparoscopic Surgery” by Atkinson, Giraud, Togioka, Jones and Cigarroa, Feb. 14, 2017 [Circulation. 2017; 135:700-710. DOI: 10.1161/CIRCULATIONAHA.116.023262].

Accordingly, it is important that the rate of insufflating of a cavity, vessel, lumen or organ and in particular, the peritoneal cavity, should not exceed a safe insufflating rate, particularly, during a laparoscopic procedure being carried out in the peritoneal cavity, and it is also important that the volume of the cavity should not exceed a predefined maximum volume, and further, it is important that the heart rate and/or the blood pressure of a subject should not fall below minimum predefined values during insufflating of the cavity of the subject, and it is also preferable that the heart rate or the blood pressure of a subject should not exceed maximum predefined values during insufflating of the cavity.

The present invention is directed towards an insufflator which addresses at least some of these problems. The invention is also directed towards a method for insufflating a cavity, vessel, lumen or organ in the body of a human or animal subject which addresses at least some of these problems.

Throughout this specification, the term “cavity” is used to include any cavity, vessel, lumen or organ in the body of a human or animal subject which may be insufflated.

### SUMMARY OF THE INVENTION

According to the invention there is provided an insufflator for insufflating a cavity in the body of a human or animal subject, the insufflator comprising:
a flow controller for controlling delivery of insufflating gas to the cavity,
an electronic memory storing a reference value of a maximum rate of insufflating the cavity,
a monitoring means for monitoring insufflating of the cavity and for producing a signal indicative of the insufflating of the cavity, and
a signal processor, the signal processor being programmed
   to read the signal produced by the monitoring means indicative of the insufflating of the cavity,
   to determine the rate at which the cavity is being insufflated from the signal read from the monitoring means,
   to compare the determined rate at which the cavity is being insufflated with the stored reference value, and
   to control the flow controller to prevent the rate at which the cavity is being insufflated exceeding the stored reference value.

In one embodiment of the invention the signal processor is programmed to control the flow controller to reduce the flow rate at which insufflating gas is being delivered to the cavity or to pause delivery of insufflating gas to the cavity if the determined rate at which the cavity is being insufflated exceeds the stored reference value of the maximum rate of insufflating the cavity.

In one embodiment of the invention the reference value of the maximum rate of insufflating the cavity stored in the electronic memory is stored as a function of pressure in the cavity (cavity pressure), and preferably, the monitoring means for monitoring insufflating of the cavity comprises a pressure sensor configured to monitor the cavity pressure and to produce a signal indicative of the cavity pressure, and the signal processor is programmed to determine the rate at which the cavity is being insufflated as a function of the cavity pressure.

In one embodiment of the invention the reference value of the maximum rate of insufflating of the cavity stored in the electronic memory is stored as a function of the pressure in the cavity (cavity pressure), and preferably, as a function of one or more of the type of cavity, the sex of the subject, the age of the subject, the weight of the subject, the height of the subject, and the body mass index of the subject, and preferably, the monitoring means for monitoring insufflating of the cavity comprises a pressure sensor configured to monitor the cavity pressure and to produce a signal indicative of the cavity pressure, and the signal processor is programmed to determine the rate at which the cavity is being insufflated as a function of the cavity pressure from the signal produced by the pressure sensor, and preferably, the reference value of the maximum rate of insufflating the cavity stored in the electronic memory is stored as the maximum increase in cavity pressure per unit time, and advantageously, the signal processor is programmed to determine the increase in cavity pressure per unit time from the signal read from the pressure sensor.

In another embodiment of the invention the reference value of the maximum rate of insufflating the cavity is stored in the electronic memory as a function of the flow of the insufflating gas delivered to the cavity, and preferably, the monitoring means comprises a flow sensor for monitoring flow of insufflating gas to the cavity, and for producing a signal indicative of the flow of insufflating gas to the cavity, and the signal processor is programmed to determine the rate at which the cavity is being insufflated as a function of the flow of insufflating gas to the cavity.

In another embodiment of the invention the reference value of the reference value of the maximum rate of insufflating the cavity is stored in the electronic memory as a function of the flow of insufflating gas delivered to the cavity, and preferably, as a function of at least one of the type of cavity, the sex of the subject, the age of the subject, the weight of the subject, the height of the subject, and the body mass index of the subject, and preferably, the monitoring means comprises a flow sensor for monitoring flow of insufflating gas to the cavity, and for producing a signal indicative of the flow of insufflating gas to the cavity, and the signal processor is programmed to determine the rate at which the cavity is being insufflated as a function of the flow of insufflating gas to the cavity from the signal produced by the flow sensor, and preferably, the reference value of the maximum rate of insufflating the cavity stored in the electronic memory is stored as the maximum flow rate of insufflating gas to the cavity, and the signal processor is programmed to determine the rate of flow of insufflating gas to the cavity from the signal read from the flow sensor.

Preferably, a plurality of reference values of maximum rates of insufflating the cavity are stored in the electronic memory for respective subjects of at least one or more of different sexes, different ages, different weights, different heights, and different body mass indices, and preferably, the signal processor is programmed to determine the reference value of the maximum rate of insufflating the cavity from the electronic memory appropriate to the cavity in response to the data indicative of the one or more of the sex, the age, the weight, the height, and the body mass index of the subject inputted to the signal processor, and preferably, a plurality of the reference values are stored in the electronic memory for each one of a plurality of different types of cavities of the subject of the one or more different sexes, different ages, different weights, different heights, and different body mass indices.

In another embodiment of the invention a predefined minimum value of a predefined characteristic indicative of the performance of a heart of a subject is stored in the electronic memory and the signal processor is programmed to read a signal indicative of a characteristic of the performance of the heart of the subject corresponding to the predefined characteristic indicative of the performance of the heart of a subject, the predefined minimum value of which is stored in the electronic memory, and on the value of the signal read by the signal processor indicative of the characteristic of the performance of the heart of the subject falling below the stored predefined minimum value thereof, the signal processor is programmed to operate the flow controller to reduce or pause delivery of insufflating gas to the cavity, and/or to operate a vacuum applying means to apply a vacuum to the cavity for reducing the cavity pressure.

In one embodiment of the invention the signal processor is programmed to operate the flow controller and/or the vacuum applying means to reduce the cavity pressure until the signal read by the signal processor indicative of the characteristic of the performance of the heart of the subject commences to rise above the stored predefined minimum value, and preferably, the predefined minimum value of the predefined characteristic indicative of the performance of the heart of a subject stored in the electronic memory comprises a predefined minimum value of the heart rate of the heart of a subject.

In another embodiment of the invention a predefined maximum value of the predefined characteristic indicative of the performance of the heart of a subject is stored in the electronic memory, and on the value of the signal read by the signal processor indicative of the characteristic of the performance of the heart of the subject rising above the predefined maximum value thereof, the signal processor is programmed to in operate the flow controller to reduce or pause delivery of insufflating gas to the cavity, and/or to operate the vacuum applying means to apply a vacuum to the cavity for reducing the cavity pressure.

In a further embodiment of the invention the signal processor is programmed to operate the flow controller and/or the vacuum applying means to reduce the cavity pressure until the signal read from the signal processor indicative of the characteristic of the performance of the heart of the subject commences to fall below the predefined maximum value thereof, and preferably, the predefined maximum value of the predefined characteristic indicative of the performance of the heart of a subject stored in the electronic memory comprises a predefined maximum value of the heart rate of a subject.

In another embodiment of the invention a predefined maximum volume value is stored in the electronic memory, and the signal processor is programmed to compute the current volume of the cavity as a function of the insufflating gas delivered to the cavity, and to compare the current computed volume of the cavity with the stored predefined maximum volume value, and the signal processor is programmed to operate the flow controller to reduce or pause the flow of insufflating gas to the cavity in response to the current computed volume of the cavity exceeding the predefined maximum volume value.

The invention also provides an insufflator for insufflating a cavity in the body of a human or animal subject, the insufflator comprising:
an electronic memory storing a predefined minimum value of a predefined characteristic indicative of the performance of a heart of a subject,
a receiving means configured to receive a signal indicative of a value of a characteristic indicative of the performance of the heart of the subject, the cavity of which is being insufflated, corresponding to the predefined characteristic indicative of the performance of the heart of a subject, the predefined minimum value of which is stored in the electronic memory, and
a signal processor programmed to read the signal indicative of the value of the characteristic indicative of the performance of the heart of the subject from the receiving means, and to reduce or pause insufflating of the cavity of the subject, and/or to evacuate the cavity of the subject to reduce the cavity pressure in response to the value of the signal indicative of the characteristic of the performance of the heart of the subject falling below the predefined minimum value thereof.

Preferably, the signal processor is programmed to reduce the cavity pressure until the value of the signal read from the receiving means by the signal processor indicative of the characteristic of the performance of the heart of the subject commences to rise above the stored predefined minimum value thereof, and preferably, the predefined minimum value of the predefined characteristic indicative of the performance of the heart of a subject stored in the electronic memory comprises a predefined minimum value of the heart rate of a subject.

In another embodiment of the invention a predefined maximum value of the predefined characteristic indicative of the performance of the heart of a subject is stored in the electronic memory, and the signal processor is programmed in response to the value of the signal read from the receiving means indicative of the characteristic of the performance of the heart of the subject rising above the predefined maximum value thereof to reduce or pause insufflating of the cavity of the subject, and/or to evacuate the cavity of the subject to reduce the cavity pressure.

Preferably, the signal processor is programmed to reduce the cavity pressure until the value of the signal read from the receiving means by the signal processor indicative of the characteristic of the performance of the heart of the subject commences to fall below the predefined maximum value.

The invention also provides an insufflator for insufflating a cavity in the body of a human or animal subject, the insufflator comprising:
a flow controller for controlling delivery of insufflating gas to the cavity,
an electronic memory for storing a predefined maximum volume value for a cavity,
a flow sensor for monitoring flow of insufflating gas to the cavity and for producing a signal indicative of the flow of insufflating gas to the cavity, and
a signal processor, the signal processer being programmed
   to read the signal from the flow sensor indicative of the flow of insufflating gas to the cavity,
   to determine the volume of the cavity from the signal read from the flow sensor,
   to compare the determined volume of the cavity with the stored predefined maximum volume value for the cavity, and
   to control the flow controller to reduce or pause insufflating of the cavity in response to the computed volume of the cavity exceeding the predefined maximum volume value.

Preferably, the signal processor is programmed to read the signal from the monitoring means and/or the flow sensor at predefined time intervals, and in one embodiment of the invention each predefined time interval is of milliseconds duration, and preferably, of duration of the order of 10 milliseconds.

In another embodiment of the invention each predefined time interval lies in the range of 1 second to 3 seconds, and preferably, lies in the range of 1 second to 2 seconds, and ideally, each predefined time interval is approximately 1.5 seconds. In one embodiment of the invention insufflating of the cavity is paused during reading of the signal from the monitoring means, and preferably, the insufflating of the cavity is paused between consecutive predefined time intervals for a predefined monitoring time period in the range 0.25 seconds to 0.75 seconds during which the signal processor reads the signal from the monitoring means, and advantageously, each predefined monitoring time period is approximately 0.5 seconds.

Preferably, the reference value of the maximum rate of insufflating the cavity is stored in the electronic memory as the value of the maximum increase in cavity pressure per unit time.

In another embodiment of the invention the signal processor is programmed to determine the rate of insufflating of the cavity as a function of the cavity pressure, and preferably, the signal processor is programmed to determine the rate at which the cavity is being insufflated as the increase in the cavity pressure per unit time.

In another embodiment of the invention the signal processor is programmed to compute the increase of the cavity pressure per unit time from the signal read from the pressure sensor.

In one embodiment of the invention the signal processor is programmed to compare the computed increase in cavity pressure per unit time with the stored reference value thereof.

Preferably, the reference value of the maximum rate of insufflating the cavity is stored in the electronic memory as a value of the maximum rate at which insufflating gas may be delivered to the cavity.

In another embodiment of the invention the monitoring means comprises a flow sensor for monitoring flow of insufflating gas to the cavity, and for producing a signal indicative of the flow rate of insufflating gas to the cavity.

In one embodiment of the invention the signal processor is programmed to determine the rate at which the cavity is being insufflated as a function of the flow rate at which insufflating gas is being delivered to the cavity from the signal read from the flow sensor, and preferably, the signal processor is programmed to determine the flow rate at which the insufflating gas is being delivered to the cavity from the signal read from the flow sensor.

In another embodiment of the invention the signal processor is programmed to compare the determined flow rate at which insufflating gas is being delivered to the cavity with the stored reference value thereof.

In one embodiment of the invention a plurality of reference values are stored in the electronic memory, and preferably, at least one reference value is provided for each one of a plurality of different types of cavities of a subject.

In one embodiment of the invention the reference value of the maximum rate of insufflating the cavity is stored as a function of the type of the cavity.

In another embodiment of the invention the reference value of the maximum rate of insufflating the cavity is stored as a function of the type of subject.

In another embodiment of the invention the reference value of the maximum rate of insufflating the cavity is stored as a function of the sex of the subject.

In another embodiment of the invention the reference value of the maximum rate of insufflating the cavity is stored as a function of the age of the subject.

In another embodiment of the invention the reference value of the maximum rate of insufflating the cavity is stored as a function of the body mass index of the subject.

In another embodiment of the invention the reference value of the maximum rate of insufflating the cavity is stored as a function of the weight of the subject.

In another embodiment of the invention the reference value of the maximum rate of insufflating the cavity is stored as a function of the height of the subject.

In another embodiment of the invention the reference value of the maximum rate of insufflating the cavity is stored for a human subject.

In another embodiment of the invention the reference value of the maximum rate of insufflating the cavity is stored for an animal subject.

In another embodiment of the invention a plurality of reference values are stored in the electronic memory for each type of cavity, at least some of the reference values for each type of cavity may relate to subjects of different ages or within different age ranges. In another embodiment of the invention at least some of the reference values for each type of cavity may relate to subjects of different weights or within different weight ranges. In another embodiment of the invention at least some of the reference values for each type of cavity may relate to subjects of different heights or within different height ranges. In a further embodiment of the invention at least some of the reference values for each type of cavity may relate to subjects of different body mass indices or within different body mass index ranges. In one embodiment of the invention at least some of the reference values for each type of cavity may relate to the sex of the subject.

Preferably, the stored reference values of the maximum rate of insufflating the cavity are stored and cross-referenced with the type or types of cavity, and at least one or more of the different ages of subjects, the different age ranges of subjects, the different weights or different weight ranges of subjects, the different heights or height ranges of subjects, the different body mass indices or body mass index ranges of subjects, or the different sexes of subjects.

In another embodiment of the invention the signal processor is programmed to control the flow controller to prevent the rate at which the cavity is being insufflated exceeding the stored reference value of the maximum rate of insufflating the cavity in response to the cavity being insufflated to a predefined volume or to a predefined pressure.

In one embodiment of the invention the predefined volume of the cavity comprises a predefined proportion of the maximum volume to which the cavity may be safely insufflated.

In another embodiment of the invention the predefined pressure of the cavity comprises a predefined proportion of the maximum pressure to which the cavity may be safely insufflated.

In one embodiment of the invention the predefined proportion of the maximum volume to which the cavity may be safely insufflated lies in the range of 40% to 80% of the maximum volume to which the cavity may be safely insufflated.

In another embodiment of the invention the predefined proportion of the maximum volume to which the cavity may be safely insufflated lies in the range of 50% to 75% of the maximum volume to which the cavity may be safely insufflated, and preferably, lies in the range of 60% to 70% of the maximum volume to which the cavity may be safely insufflated.

In another embodiment of the invention the predefined pressure of the cavity comprises a predefined proportion of the maximum pressure to which the cavity may be safely insufflated.

In one embodiment of the invention the predefined proportion of the maximum pressure to which the cavity may be safely insufflated lies in the range of 40% to 80% of the maximum pressure to which the cavity may be safely insufflated.

In another embodiment of the invention the predefined proportion of the maximum pressure to which the cavity may be safely insufflated lies in the range of 50% to 75% of the maximum pressure to which the cavity may be safely insufflated, and preferably, lies in the range of 60% to 70% of the maximum pressure to which the cavity may be safely insufflated.

In another embodiment of the invention the predefined volumes or the predefined pressures are stored in the electronic memory and cross-referenced with the type or types of cavity and at least one or more of the different ages or age ranges of the subjects, the different weights or different weight ranges of the subjects, the different heights or different height ranges of the subjects, the different body mass indices or different body mass index ranges of the subjects or the different sexes of the subjects.

In one embodiment of the invention the reference value of the maximum rate of insufflating the cavity may be progressively reduced from the stored reference value thereof as insufflating of the cavity to the target pressure proceeds.

In one embodiment of the invention the progressive reduction in the reference value of the maximum rate of insufflating the cavity may commence from commencement of insufflating of the cavity, or from the cavity pressure reaching or being above the predefined pressure or the volume of the cavity reaching or being above the predefined volume.

In one embodiment of the invention the rates at which the reference maximum rate of insufflating of the cavity is to be progressively reduced is stored and cross-referenced with the cavity or the type of cavity, and with one or more of the different ages or age ranges of subjects, the different weights or the different weight ranges of subjects, the different heights or height ranges of subjects, the different body mass indices or body mass index ranges of subjects, or the different sexes of subjects.

In another embodiment of the invention the signal processor is programmed to progressively reduce the stored appropriate reference value of the maximum rate of insufflating the cavity by the appropriate stored predefined rate at which the reference value of the maximum rate of insufflating of the cavity is to be progressively reduced from the commencement of insufflating of the cavity or from the cavity being insufflated to the predefined pressure or to the predefined volume, and preferably, the signal processor is programmed to control the flow controller to reduce the flow rate of insufflating gas to the cavity to progressively reduce the stored appropriate reference value of the maximum rate of insufflating the cavity corresponding to the current cavity pressure or the current cavity volume.

In one embodiment of the invention the insufflator is adapted for applying a vacuum to the cavity for withdrawing insufflating gas therefrom.

Preferably, the insufflator is adapted for applying a vacuum to the cavity for withdrawing insufflating gas therefrom or for ceasing delivery of insufflating gas to the cavity in response to one of the cavity pressure exceeding a predefined maximum cavity pressure, or the volume value of the cavity exceeding a predefined maximum cavity volume value.

In one embodiment of the invention the predefined maximum cavity pressure value is stored in the electronic memory or within the signal processor, and in another embodiment of the invention the predefined maximum cavity volume value is stored in the electronic memory or within the signal processor.

In one embodiment of the invention the stored predefined maximum cavity volume value is stored as a function of a volume of insufflating gas delivered to the subject, and preferably, the stored predefined maximum cavity volume value is stored as the total volume of insufflating gas delivered to the cavity from the commencement of insufflating thereof.

In one embodiment of the invention the signal processor is adapted to determine the volume of the cavity as a function of the flow rate of the insufflating gas delivered to the cavity, and preferably, the signal processor is programmed to determine the volume of the cavity from the flow rate of insufflating gas delivered to the cavity multiplied by the time duration during which the insufflating gas is delivered to the cavity.

In another embodiment of the invention the signal processor is programmed to determine the volume of the cavity as the total volume of insufflating gas delivered to the cavity from the commencement of insufflating thereof less the volume of insufflating gas leaking from the cavity from the commencement of insufflating of the cavity.

In one embodiment of the invention a plurality of maximum volume values are stored in the electronic memory for each type of cavity, and at least some of the maximum volume values for each type of cavity may relate to subjects of different ages or within different age ranges. In another embodiment of the invention at least some of the maximum volume values for each type of cavity may relate to subjects of different weights or within different weight ranges. In another embodiment of the invention at least some of the maximum volume values for each type of cavity may relate to subjects of different heights or within different height ranges. In a further embodiment of the invention at least some of the maximum volume values for each type of cavity may relate to subjects of different body mass indices or within different body mass index ranges. In one embodiment of the invention at least some of the reference values for each type of cavity may relate to the sex of the subject.

Preferably, the stored maximum volume values are stored and cross-referenced with the type or types of cavity and at least one or more of the different ages or different age ranges of the subjects, the different weights or different weight ranges of the subjects, the different heights or different height ranges of the subjects, the different body mass indices or body mass index ranges of the subjects or the different sexes of the subjects.

In one embodiment of the invention the predefined maximum value of the predefined characteristic indicative of a performance value of a heart of a subject, above which the performance of the heart should not exceed, is stored in the electronic memory or within the signal processor.

In another embodiment of the invention the predefined minimum value of the predefined characteristic indicative of a performance value below which the performance of a heart of a subject should not fall, is stored in the electronic memory or within the signal processor.

In another embodiment of the invention the signal processor is programmed to read a signal indicative of a characteristic of the performance of the heart of a subject, corresponding to the stored predefined maximum value of the predefined characteristic and/or the stored predefined minimum value of the predefined characteristic thereof, and on the value of the signal indicative of the performance characteristic of the heart of the subject exceeding the stored predefined maximum value of the predefined characteristic or falling below the stored predefined minimum value of the characteristic, the signal processor is programmed to operate the flow controller either to pause insufflating of the cavity and/or to apply the vacuum to the cavity for withdrawing insufflating gas therefrom.

In one embodiment of the invention the predefined maximum value of the predefined characteristic stored comprises a predefined maximum value of the heart rate of a subject, and in another embodiment of the invention the predefined minimum value of the predefined characteristic comprises a predefined minimum value of the heart rate of a subject.

In one embodiment of the invention the predefined characteristic indicative of the performance of a heart of a subject comprises the blood pressure of a subject, and preferably, a predefined minimum value of the blood pressure of a subject is stored, and preferably, the stored predefined minimum value of the blood pressure of a subject is stored as a minimum value of the systolic blood pressure of a subject, and advantageously, is stored as a predefined minimum value of the diastolic blood pressure of a subject.

In one embodiment of the invention the predefined characteristic indicative of the performance of a heart of a subject is stored as a predefined maximum value of the blood pressure of a subject, and preferably, the predefined maximum value of the blood pressure of a subject is stored as a maximum value of the systolic blood pressure of a subject, and advantageously, is stored as a predefined maximum value of the diastolic blood pressure value of a subject.

Preferably, the receiving means is configured to receive a signal indicative of the blood pressure of the subject, and advantageously, the receiving means is adapted to receive a signal indicative of the systolic blood pressure of the subject, and advantageously, the receiving means is adapted for receiving a signal indicative of the diastolic blood pressure value of the subject.

In one embodiment of the invention the insufflator comprises a vacuum pump.

In an alternative embodiment of the invention the insufflator is adapted for receiving a vacuum from an external vacuum source, and the insufflator comprises an isolating valve for selectively connecting the external vacuum to the cavity for withdrawing insufflating gas therefrom.

In another embodiment of the invention the isolating valve is operable by the signal processor in response to the signal indicative of the characteristic of the performance of the heart of a subject exceeding the predefined maximum value of the predefined characteristic, or falling below the predefined minimum value of the predefined characteristic.

In another embodiment of the invention the signal processor is responsive to the read signal indicative of the performance of the heart exceeding the predefined maximum value of the predefined characteristic or falling below the predefined minimum value of the predefined characteristic for operating the flow controller to terminate delivery of insufflating gas to the cavity, or for applying a vacuum to the cavity for withdrawing insufflating gas therefrom.

In another embodiment of the invention the signal processor is programmed to read the signal of the performance of the heart of a subject as the heart rate thereof.

In another embodiment of the invention a means for monitoring a performance characteristic of the heart of a subject is provided, and is configured to produce a signal indicative of the performance characteristic of the subject to the signal processor.

In one embodiment of the invention the signal indicative of the performance characteristic of the subject is wirelessly transmitted to the signal processor, and in an alternative embodiment of the invention the signal indicative of the performance characteristic of the subject is transmitted to the signal processor by hard wiring.

In a further embodiment of the invention the means for monitoring the performance characteristic of the heart of a subject is configured to monitor the heart rate of the subject.

In another embodiment of the invention the signal processor is responsive to the determined rate at which the cavity is being insufflated exceeding the stored reference value of the maximum rate of insufflating the cavity for applying the vacuum to the cavity for withdrawing insufflating gas from the cavity to reduce the pressure therein.

In one embodiment of the invention an interface means is provided for inputting data to the signal processor.

In one embodiment of the invention the interface means is adapted for inputting data relating to the type of the cavity to be insufflated to the signal processor.

In another embodiment of the invention the interface means is adapted for inputting data relating to a subject to the signal processor.

In another embodiment of the invention the interface means is adapted for inputting data relating to the sex of the subject to the signal processor.

In another embodiment of the invention the interface means is adapted for inputting data relating to the age of the subject to the signal processor.

In another embodiment of the invention the interface means is adapted for inputting data relating to the weight of the subject to the signal processor.

In another embodiment of the invention the interface means is adapted for inputting data relating to the height of the subject to the signal processor.

Preferably, the interface means is adapted for inputting data relating to the body mass index of the subject to the signal processor.

In another embodiment of the invention the signal processor is programmed to read data inputted through the interface means, and preferably, the signal processor is programmed to determine the appropriate reference value from the reference values stored in the electronic memory in response to the data entered to the signal processor through the interface means relating to the subject and the cavity of which is to be insufflated, and to compare the determined value of the rate at which the cavity is being insufflated determined from the signal read from the monitoring means with the appropriate reference value during insufflating of the cavity, and preferably, the signal processor is programmed to store the appropriate reference value in a memory of the signal processor.

In one embodiment of the invention the signal processor is programmed to determine the appropriate reference value from the electronic memory prior to the commencement of insufflating of the cavity.

In one embodiment of the invention the signal processor is programmed to determine the appropriate maximum volume value from the maximum volume values stored in the electronic memory in response to the data entered to the signal processor through the interface means relating to the subject and the cavity of which is to be insufflated, and to compare the volume of the cavity determined from the signal read from flow sensor with the appropriate maximum volume value during insufflating of the cavity, and preferably, the signal processor is programmed to store the appropriate reference value in a memory of the signal processor.

Further the invention provides a method for insufflating a cavity in the body of a human or animal subject, the method comprising:
delivering insufflating gas to the cavity,
monitoring insufflating of the cavity,
determining the rate of insufflating of the cavity,
comparing the determined rate of insufflating of the cavity with a stored reference value of a maximum rate of insufflating the cavity, and
controlling the rate of insufflating of the cavity to prevent the rate at which the cavity is being insufflated exceeding the stored reference value.

In one embodiment of the invention the rate at which the cavity is being insufflated is reduced or insufflating of the cavity is paused if the determined rate of insufflating of the cavity exceeds the stored reference value of the maximum rate of insufflating the cavity.

In another embodiment of the invention the reference value of the maximum rate of insufflating the cavity is stored as a function of cavity pressure, and preferably, the cavity pressure is monitored, and the rate of insufflating of the cavity is determined as a function of the cavity pressure.

In one embodiment of the invention the reference value of the maximum rate of insufflating the cavity is stored as a function of cavity pressure, and preferably as a function of one or more of the type of cavity, the sex of the subject, the age of the subject, the weight of the subject, the height of the subject, and the body mass index of the subject, and preferably, the cavity pressure is monitored, and the rate of insufflating of the cavity is determined as a function of the cavity pressure, and advantageously, the reference value of the maximum rate of insufflating the cavity is stored as a function of the maximum increase in cavity pressure per unit time, and advantageously, the increase in the cavity pressure per unit time of the subject is compared with the reference value of the maximum increase in cavity pressure per unit time.

In another embodiment of the invention the reference value of the maximum rate of insufflating the cavity is stored as a function of the flow rate of insufflating gas delivered to the cavity, and preferably, the flow of insufflating gas to the cavity is monitored, and the rate at which the cavity is being insufflated is determined as a function of the flow rate of insufflating gas to the cavity.

In one embodiment of the invention the reference value of the maximum rate of insufflating the cavity is stored as a function of the flow rate of the insufflating gas delivered to the cavity, and preferably, as a function of one or more of the type of cavity, the sex of the subject, the age of the subject, the weight of the subject, the height of the subject, and the body mass index of the subject, and preferably, the flow of insufflating gas to the cavity is monitored, and the rate at which the cavity is being insufflated is determined as the flow rate of insufflating gas to the cavity, and advantageously, the reference value of the maximum rate of insufflating the cavity is stored as the maximum flow rate of insufflating gas to the cavity, and preferably, the determined flow rate of insufflating gas to the cavity is compared with the reference value of the maximum flow rate of insufflating gas.

Preferably, a plurality of reference values of maximum rates of insufflating the cavity are stored for respective subjects of at least one or more of different sexes, different ages, different weights, different heights, and different body mass indices, and preferably, the reference value of the maximum rate of insufflating the cavity appropriate to the cavity is determined from the stored reference values thereof from data indicative of the one or more of the sex, the age, the weight, the height, and the body mass index of the subject, and preferably, a plurality of the reference values are stored for each one of a plurality of different types of cavities for the subjects of the one or more different sexes, different ages, different weights, different heights, and different body mass indices.

In one embodiment of the invention the stored reference values of the maximum rate of insufflating the cavity are stored and cross-referenced with the type or types of cavity, and at least one or more of the different age or different age ranges of subjects, the different weight or weight ranges of subjects, the different heights or height ranges of subjects, the different body mass indices or body mass index ranges of subjects or the different sexes of subjects.

In another embodiment of the invention a predefined minimum value of a predefined characteristic of the performance of the heart of a subject is stored, and a characteristic of the performance of the heart of the subject is monitored corresponding to the predefined characteristic indicative of the performance of the heart of a subject, the predefined minimum value of which is stored, and on the value of the monitored characteristic of the performance of the heart of the subject falling below the stored predefined minimum value thereof, the insufflating of the cavity is reduced or paused and/or a vacuum is applied to the cavity to reduce the cavity pressure.

In another embodiment of the invention the flow of insufflating gas to the cavity is reduced or paused and/or a vacuum is applied to the cavity until the value of the monitored characteristic of the performance of the heart of the subject commences to rise above the stored predefined minimum value thereof, and preferably, the stored predefined minimum value of the predefined characteristic indicative of the performance of the heart of a subject comprises a predefined minimum value of the heart rate of the heart of a subject.

In one embodiment of the invention a predefined maximum value of the predefined characteristic indicative of the performance of the heart of a subject is stored, and on the value of the monitored characteristic of the performance of the heart of the subject rising above the stored predefined maximum value thereof, the flow of insufflating gas to the cavity is reduce or paused and/or a vacuum is applied to the cavity to reduce the cavity pressure.

In another embodiment of the invention the flow of insufflating gas to the cavity is reduced or paused and/or a vacuum is applied to the cavity until the value of the monitored characteristic of the performance of the heart of the subject commences to fall below the stored predefined maximum value thereof, and preferably, the stored predefined maximum value of the predefined characteristic of the performance of the heart of a subject comprises a predefined maximum value of the heart rate of the heart of a subject.

In a further embodiment of the invention the volume of the cavity is computed as a function of the insufflating gas delivered to the cavity, and if the computed volume of the cavity exceeds a predefined maximum volume value, insufflating of the cavity is reduced or paused.

The invention also provides a method for insufflating a cavity in the body of a human or animal subject, the method comprising:
storing a predefined minimum value of a predefined characteristic indicative of the performance of a heart of a subject,
monitoring a value of a characteristic indicative of the performance of the heart of the subject, the cavity of which is being insufflated corresponding to the predefined characteristic indicative of the performance of the heart of a subject, the predefined minimum value of which is stored, and
on the value of the signal indicative of the characteristic of the performance of the heart of the subject falling below the predefined minimum value thereof reducing or pausing insufflating of the cavity of the subject, and/or evacuating the cavity of the subject to reduce the cavity pressure.

In one embodiment of the invention the cavity pressure is reduced until the value of the signal read by the signal processor indicative of the characteristic of the performance of the heart of the subject commences to rise above the stored predefined minimum value thereof, and preferably, the predefined minimum value of the predefined characteristic indicative of the performance of the heart of a subject is stored as a predefined minimum value of the heart rate of a subject.

In another embodiment of the invention a predefined maximum value of the predefined characteristic indicative of the performance of the heart of a subject is stored, and on the value of the received signal indicative of the characteristic of the performance of the heart of the subject rising above the predefined maximum value thereof insufflating of the cavity of the subject is reduced or paused, and/or the cavity of the subject is evacuated to depressurise the cavity.

In another embodiment of the invention the cavity pressure is reduced until the value of the signal indicative of the characteristic of the performance of the heart of the subject commences to fall below the maximum predefined value thereof.

Additionally, the invention provides a method for insufflating a cavity in the body of a human or animal subject, the method comprising:
delivering insufflating gas to the cavity,
monitoring the flow of insufflating gas to the cavity,
determining the volume of the cavity as a function of the flow of insufflating gas to the cavity,
comparing the determined volume of the cavity with a stored predefined maximum volume value, and
controlling insufflating of the cavity to reduce the volume of the cavity if the determined volume of the cavity exceeds the predefined maximum volume value.

In one embodiment of the invention the rate at which insufflating gas is being delivered to the cavity is reduced or is paused if the determined rate of insufflating of the cavity exceeds the stored reference value.

In one embodiment of the invention the stored reference value of the maximum rate of insufflating the cavity is stored as a function of the cavity pressure, and preferably, the stored reference value of the maximum rate of insufflating the cavity is stored as a value of the maximum increase in cavity pressure per unit time.

In another embodiment of the invention the cavity pressure is monitored.

In another embodiment of the invention the rate of insufflating of the cavity is determined as a function of the cavity pressure, and preferably, the rate at which the cavity is being insufflated is determined as the increase in the cavity pressure per unit time.

In another embodiment of the invention the increase of the cavity pressure per unit time is computed from the cavity pressure.

In one embodiment of the invention the computed increase in cavity pressure per unit time is compared with the stored reference value thereof.

In another embodiment of the invention the reference value of the maximum rate of insufflating the cavity is stored as a function of the flow rate of the insufflating gas delivered to the cavity, and preferably, the reference value of the maximum rate of insufflating the cavity is stored as a maximum flow rate of insufflating gas to the cavity.

In another embodiment of the invention the flow of insufflating gas to the cavity is monitored.

In one embodiment of the invention the rate at which the cavity is being insufflated is determined as a function of the flow rate of insufflating gas to the cavity, and preferably, the flow rate at which the insufflating gas is being delivered to the cavity is computed from the flow of insufflating gas to the cavity.

In another embodiment of the invention the computed flow rate at which insufflating gas is being delivered to the cavity is compared with the stored reference value thereof.

In one embodiment of the invention a plurality of reference values are stored, and preferably, at least one reference value is stored for each one of a plurality of different types of cavities of a subject.

In another embodiment of the invention a plurality of reference values are stored in the electronic memory for each type of cavity, at least some of the reference values for each type of cavity may relate to subjects of different ages or within different age ranges. In another embodiment of the invention at least some of the reference values for each type of cavity may relate to subjects of different weights or within different weight ranges. In another embodiment of the invention at least some of the reference values for each type of cavity may relate to subjects of different heights or within different height ranges. In a further embodiment of the invention at least some of the reference values for each type of cavity may relate to subjects of different body mass indices or within different body mass index ranges. In one embodiment of the invention at least some of the reference values for each type of cavity may relate to the sex of the subject.

Preferably, the stored reference values of the maximum rate of insufflating the cavity are stored and cross-referenced with the type or types of cavity, and at least one or more of the different ages or different age ranges of subjects, the different weights or weight ranges of subjects, the different heights or height ranges of subjects, the different body mass indices or body mass index ranges of subjects, or the different sexes of subjects.

In one embodiment of the invention the method further comprises applying a vacuum to the cavity for withdrawing insufflating gas therefrom.

Preferably, a vacuum is applied to the cavity for withdrawing insufflating gas therefrom or delivery of insufflating gas to the cavity is paused or terminated in response to one of the cavity pressure exceeding a predefined maximum cavity pressure value, or the volume of the cavity exceeding a predefined maximum cavity volume value.

In one embodiment of the invention the predefined maximum cavity volume value is defined as a function of a volume of insufflating gas delivered to the subject, and preferably, the predefined maximum cavity volume value is defined as a function of the total volume of insufflating gas delivered to the cavity from the commencement of insufflating thereof.

In one embodiment of the invention the volume of the cavity is determined as a function of the flow rate of the insufflating gas delivered to the cavity, and preferably, the volume of the cavity is determined as a function of the flow rate of insufflating gas delivered to the cavity multiplied by the time duration during which the insufflating gas is being delivered to the cavity.

In another embodiment of the invention the volume of the cavity is determined as the total volume of insufflating gas delivered to the cavity from the commencement of insufflating thereof less the volume of insufflating gas leaking from the cavity from the commencement thereof.

In one embodiment of the invention a performance value of a characteristic of a heart of a subject, a cavity of which is being insufflated, is monitored and compared with a predefined maximum value of a corresponding predefined characteristic indicative of performance of a heart of a subject, and if the monitored value exceeds the predefined maximum value of the predefined characteristic, a vacuum is applied to the cavity to withdraw insufflating gas therefrom or delivery of insufflating gas to the cavity is paused or terminated.

In another embodiment of the invention the monitored performance value of the characteristic of the heart of the subject is compared with a predefined minimum value of the predefined characteristic indicative of performance of the heart of a subject, and if the monitored characteristic falls below the predefined minimum value of the predefined characteristic, a vacuum is applied to the cavity for withdrawing insufflating gas therefrom or delivery of insufflating gas to the cavity is paused or terminated.

In one embodiment of the invention the predefined maximum value of the predefined characteristic of the performance of the heart of a subject is defined as a predefined maximum value of the heart rate of a subject, and the predefined minimum value of the predefined characteristic of the performance of the heart of a subject is defined as a predefined minimum value of the characteristic of the heart of a subject.

In another embodiment of the invention a vacuum is applied to the cavity to withdraw insufflating gas therefrom, or delivery of insufflating gas to the cavity is paused or terminated in response to the determined rate at which the cavity is being insufflated exceeding the reference value of the maximum rate of insufflating the cavity.

In one embodiment of the invention the predefined minimum value of the predefined characteristic of the performance of the heart of a subject is provided as a predefined minimum value of the blood pressure of a subject, and the blood pressure of the subject is monitored and compared with the predefined minimum value of the blood pressure of a subject, and if the monitored value of the blood pressure of the subject falls below the predefined minimum value thereof, insufflating of the cavity of the subject is paused or terminated, and/or the cavity of the subject is evacuated.

Preferably, if the monitored value of the characteristic of performance of the heart of the subject, or the blood pressure of the subject does not rise above the predefined minimum value of the characteristic or the predefined minimum value of the blood pressure within a first predefined time period, the cavity of the subject is evacuated.

In another embodiment of the invention the predefined maximum value of the predefined characteristic of performance of the heart of a subject is provided as a predefined maximum value of the blood pressure of a subject, and the monitored blood pressure of the subject is compared with the predefined minimum value of the blood pressure of a subject, and if the monitored blood pressure value exceeds the predefined maximum value thereof, insufflating of the cavity is paused or terminated, and/or the cavity is evacuated.

Preferably, if the monitored characteristic of performance of the heart of the subject or the blood pressure of the subject does not fall below the predefined maximum value of the characteristic or the blood pressure within a second predefined time period, the cavity of the subject is evacuated.

In one embodiment of the invention the signal processor is programmed to compare the signal indicative of the blood pressure of the subject with a stored predefined maximum value of the blood pressure of a subject, and if the signal indicative of the blood pressure of the subject exceeds the stored predefined maximum value of the blood pressure, the signal processor is programmed to pause or terminate delivery of insufflating gas to the subject, and preferably, if the signal indicative of the blood pressure of the subject does not fall below the stored predefined maximum value of the blood pressure within the second predefined time period, the signal processor is programmed to evacuate the cavity of the subject.

In one embodiment of the invention the signal processor is programmed to compare the signal indicative of the blood pressure of the subject with a stored predefined minimum value of the blood pressure of a subject, and if the signal indicative of the blood pressure of the subject falls below the stored predefined minimum value of the blood pressure, the signal processor is programmed to terminate delivery of insufflating gas to the subject, and preferably, if the signal indicative of the blood pressure of the subject does not rise above the stored predefined minimum value of the blood pressure within the first predefined time period, the signal processor is programmed to evacuate the cavity of the subject.

In one embodiment of the invention the first and second predefined time periods may be the same or different.

In another embodiment of the invention each of the first and second time periods lie in the range of 10 seconds to 60 seconds, and preferably, lie in the range of 15 seconds to 50 seconds, and advantageously, each of the first and second predefined time periods is approximately 30 seconds.

The invention also provides a method for insufflating a cavity in the body of a human or animal subject, the method comprising monitoring a characteristic of performance of the heart of a subject, or the blood pressure of a subject, and controlling the flow rate of insufflating gas to the cavity of the subject, terminating delivery of insufflating gas to the cavity of the subject, and/or evacuating the cavity of the subject to depressurise the cavity in response to the characteristic of the performance of the heart of the subject, or the blood pressure of the subject.

### Advantages of the Invention

The advantages of the invention are many. A particularly important advantage of the invention is that any risk of bradycardia, hypotension and cardiac arrest during insufflating of a cavity in the body of a human or animal subject, and in particular, insufflating the peritoneal cavity in the body of a human or animal subject, is substantially eliminated. This is achieved by virtue of the fact that by monitoring the rate at which the cavity is being insufflated by either monitoring the increase in cavity pressure per unit time during insufflating thereof, or by monitoring the rate of flow of insufflating gas to the cavity during insufflating thereof, and by preventing the cavity being insufflated at a rate at which the increase in cavity pressure per unit time or the rate of flow of insufflating gas to the cavity of the subject exceeds a predefined maximum increase in cavity pressure per unit time, or a maximum increase in the rate of flow of insufflating gas to the cavity dependent on one or more of the sex of the subject, the age of the subject and the body mass index of the subject, the cavity may be insufflated to a desired pressure, while at the same time avoiding the risk of bradycardia, hypotension and cardiac arrest.

A further advantage of the invention is achieved when the heart rate of the subject is monitored, in that should the heart rate of the subject exceed a predefined maximum heart rate value or fall below a predefined minimum heart rate value, insufflating of the cavity may be terminated, thereby avoiding any risk of cardiac arrest. This advantage is particularly important in that if the heart rate of the subject should fall below a predefined minimum heart rate value, which could lead to cardiac arrest, insufflating of the cavity is terminated, thereby avoiding cardiac arrest.

A further advantage of the invention is achieved by preventing the volume to which the cavity is being insufflated exceeding a predefined maximum volume value, and by relating the predefined maximum volume value to the compliance of the cavity being insufflated, and in particular, by relating the predefined maximum volume value to one of more of the sex of the subject, the age of the subject and the body mass index of the subject, in that any danger of over distending the cavity during insufflating thereof is avoided.

The invention will be more clearly understood from the following description of some preferred embodiments thereof which are given by way of example only with reference to the accompanying drawings.

### BRIEF DESCRIPTION OF THE DRAWINGS

FIG. 1 is a block representation of an insufflator according to the invention,

FIG. 2 illustrates a look-up table of data stored in an electronic memory of the insufflator of FIG. 1,

FIG. 3 is a block representation of an insufflator according to another embodiment of the invention,

FIG. 4 illustrates a look-up table of data stored in an electronic memory of the insufflator 1 of FIG. 3,

FIG. 5 is a block representation of an insufflator according to another embodiment of the invention, and

FIG. 6 illustrates a look-up table of data stored in an electronic memory of the insufflator of FIG. 5.

### DETAILED DESCRIPTION OF THE INVENTION

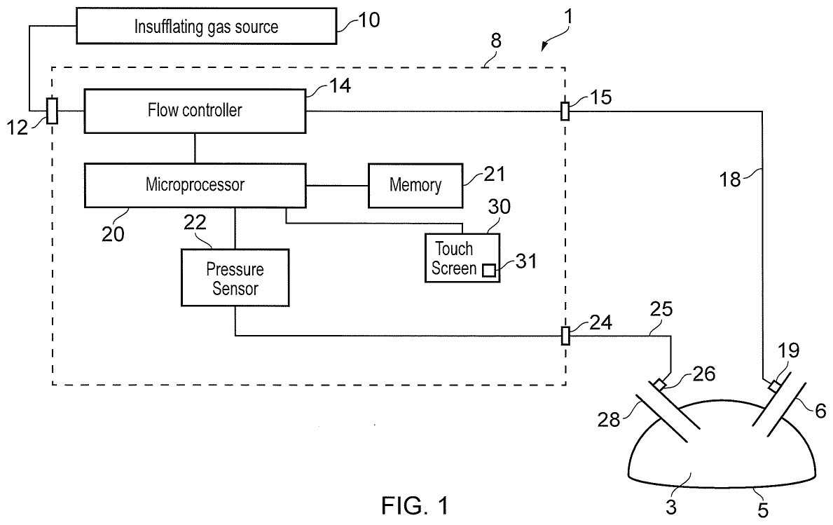

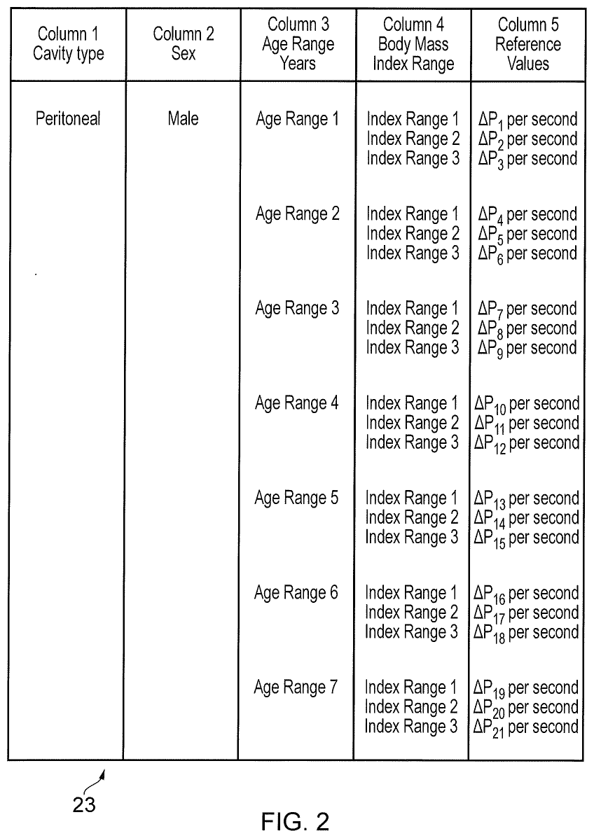

Referring to the drawings and initially to FIGS. 1 and 2 thereof, there is illustrated an insufflator according to the invention indicated generally by the reference numeral 1 for insufflating a cavity in the body of a human or animal subject. In this embodiment of the invention the insufflator is adapted for insufflating the peritoneal cavity 3 in the body 5 of a human subject through a trocar 6 during a laparoscopic procedure being carried out in the peritoneal cavity 3.

The insufflator 1 comprises a housing 8 and is configured in this embodiment of the invention to receive insufflating gas from an external source 10 thereof, which typically, would be a source of pressurised carbon dioxide which is readily available in a hospital operating theatre or in any other hospital location in which the insufflator 1 would be used. An inlet port 12 is provided in the housing 8 for connecting the insufflator 1 to the insufflating gas source 10. However, it is envisaged that in some embodiments of the invention the insufflator 1 may comprise an internal pressurised source of carbon dioxide or other suitable insufflating gas which would be contained within a pressurised container located within the housing 8.

A flow controller 14 is located in the housing connected to the inlet port 12 for receiving pressurised insufflating gas from the insufflating gas source 10. The flow controller 14 controls delivery of insufflating gas from the insufflating gas source to the cavity 3 through a first outlet port 15 in the housing 8. A first gas line comprising an elongated first conduit 18 connected to the first outlet port 15 and to the trocar 6 accommodates insufflating gas from the first outlet port 15 to the cavity 3. The first conduit 18 is connected to an insufflating gas inlet port 19 in the trocar 6, as will be well known to those skilled in the art.

A signal processor, in this embodiment of the invention a microprocessor 20 located in the housing 8 controls the operation of the insufflator 1 and operates and controls the operation of the flow controller 14 for delivering insufflating gas to the cavity 3, and in particular, for preventing the rate of insufflating of the cavity 3 exceeding a maximum rate of insufflating of the cavity as will be described in detail below.

An electronic memory 21 located in the housing 8, which may be a stand-alone memory 21, such as a read only memory or a random access memory, or may be a memory incorporated in the microprocessor 20, stores data accessible to the microprocessor 20 required for the operation of the insufflator 1. In particular, the electronic memory 21 stores a plurality of reference values of maximum rates of insufflating different cavities in the bodies of different human subjects above which rates of insufflating, bradycardia, hypotension or cardiac arrest could occur.

The reference values of the maximum rates of insufflating the different cavities in this embodiment of the invention stored in the memory 21 are stored in the form of a plurality of look-up tables, one of which is the look-up table 23 is illustrated in FIG. 2, which will be described in detail below. Briefly, reference values of the maximum rates of insufflating different types of cavities in the body of a human subject are stored in the look-up tables, and a plurality of reference values of maximum rates of insufflating each type of cavity are also stored in the look-up tables, for different types of subjects, for example, male and female subjects, and male and female subjects of respective different age ranges and respective different body mass index ranges. This, as discussed above, will be described in more detail below with reference to FIG. 2.

A monitoring means for monitoring insufflating of the cavity 3 in this embodiment of the invention comprises a pressure sensor 22 located in the housing 8 and is connected to a second outlet port 24 in the housing 8. A second gas line comprising an elongated second conduit 25 connected to the second outlet port 24 and to an insufflating gas inlet port 26 of a trocar 28, which extends into the cavity 3, communicates the pressure sensor 22 with the cavity 3 for monitoring the pressure in the cavity 3 (cavity pressure). The pressure sensor 22 is configured to produce a signal indicative of the cavity pressure. The microprocessor 20 is programmed to read the signal produced by the pressure sensor 22 at predefined time intervals, typically of ten milliseconds and to determine the rate of insufflating of the cavity 3 as a function of the cavity pressure, as will be described below.

If however, a second gas line to the cavity 3 of the subject is not available, the cavity pressure would be monitored through the first conduit 18. In which case, the predefined time intervals at which the microprocessor 20 would read the signal from the pressure sensor 22 would be in the order of 1.5 seconds, although, the predefined time intervals may range from 1 second to 3 seconds. Insufflating of the cavity between the consecutive predefined time intervals would be paused for predefined monitoring time periods of approximately 0.5 seconds, in order to allow the cavity pressure to stabilise, so that the signal read from the pressure sensor 22 by the microprocessor 20 would be indicative of the cavity pressure.

An interface means, which may be any suitable interface, for example, a touchscreen, a keypad, a voice recognition unit, or other such interface, in this case comprises a touchscreen 30, which is located on the housing 8 for inputting data to the microprocessor 20. The touchscreen 30 is configured to allow entry of data into the microprocessor 20, such as data relating to the type of cavity in the subject which is to be insufflated, the sex of the subject, the age of the subject and the body mass index of the subject, as well as the target pressure at which the cavity 3 is to be insufflated and other relevant data.

Referring now to FIG. 2, the reference values of the maximum rates of insufflating different cavities in human subjects of each of the male and female sexes, of different age ranges and of different body mass index ranges, as discussed above, are stored in the memory 21 in a plurality of look-up tables, one of which is the look-up table 23 of FIG. 2. The reason different reference values are stored for different cavities in the human body is due to the difference in compliance of the different cavities. Similarly, due to the difference in compliance of the same cavity in male and female subjects, and the difference in compliance of the same cavity due to age of a subject and the body mass index of a subject, different reference values of the maximum rates of insufflating the cavity are provided for each cavity for male and female subjects, and for different age ranges and different body mass index ranges of subjects.

The reference values of the maximum rates of insufflating the cavities are stored as a function of the cavity pressure. In this embodiment of the invention the reference values of the maximum rates of insufflating the cavities are stored as reference values of the maximum increases in the cavity pressure per unit time. In FIG. 2, the look-up table 23 which comprises the reference values for insufflating the peritoneal cavity in male subjects, the respective different age ranges and the respective different body mass index ranges are stored. A look-up table (not shown) in a form similar to that of the look-up table 23 of FIG. 2 is provided and stored in the memory 21 for the peritoneal cavity of female subjects which comprises reference values of the maximum increases in cavity pressure per unit time for the female subjects of respective different age ranges and of respective different body mass index ranges. The memory 21 comprises other look-up tables (not shown) of a similar format to that of the look-up table 23 of FIG. 2 which comprise reference values of the maximum increases in the cavity pressure per unit time for other cavities, for example, the stomach, the intestine, the colon, the rectum and the like, and in which look-up tables the reference values of the maximum increases in cavity pressure per unit time are stored against the sex of subjects, age ranges of subjects and body mass index ranges of subjects.

Turning now specifically to the look-up table 23 of FIG. 2, in column 1 of the look-up table 23 the cavity of subjects for which reference values are provided in Column 5 is identified as being the peritoneal cavity. Column 2 sets out the sex of the subjects for which the reference values are provided. Column 3 sets out the age ranges of the subjects for which the reference values are provided. In column 3, seven age ranges are set forth, namely, age range 1 to age range 7. Age range 1 is for male subjects of age zero years to 5 years of age. Age range 2 is for male subjects of age 6 years to 10 years, age range 3 is for male subjects of age 11 years to 15 years, age range 4 is for male subjects of age 16 years to 20 years, age range 5 is for male subjects of age 21 years to 40 years, age range 6 is for male subjects of age 41 years to 60 years, while age range 7 is for male subjects of age 61 years and greater.

Column 4 sets out three body mass index ranges against each of the seven age ranges of column 3. Body mass index range 1 includes body mass indices of underweight male subjects of body mass indices of 18.4 and below. Body mass index range 2 includes body mass indices of male subjects of normal healthy weight of body mass indices in the range of 18.5 to 24.9. Body mass index range 3 includes body mass indices of overweight, obese and morbidly obese male subjects, namely, body mass indices in the range of 25 to 40 and over. However, it is envisaged that in some embodiments of the invention more than three body mass index ranges may be provided, in particular, for subjects of age 21 and upwards. In which case, it is envisaged that the body mass index range 3 may be subdivided into three further body mass index ranges whereby one of the subdivisions of body mass index range 3 would include body mass indices for an overweight male subject but not an obese male subject whereby the body mass indices of that range would be between 25 and 29.9. A second subdivision of body mass index range 3 would include body mass indices of an obese male subject, and would include body mass indices in the range of to 39.9. A third subdivision of the body mass index range 3 would include body mass indices of a morbidly obese male subject, namely, body mass indices of 40 and over.

It is also envisaged that in the case of subjects under the age of 15, instead of providing body mass index ranges, weight ranges may be provided whereby one weight range would be a normal weight range for such subjects within the relevant age range, a second weight range would be for underweight subjects of the relevant age range, while a third weight range would be a weight range of overweight, and possibly, obese subjects of the relevant age range.

Column 5 of the look-up table 23 sets forth the reference values of the maximum increases in cavity pressure per unit time, above which the peritoneal cavity of male subjects of age within the relevant age range, and of body mass index within the relevant weight or body mass index range, should not be insufflated. The reference values in column 5 of the look-up table 23 are given as values ΔP₁ mmHg per second to ΔP₂₁ mmHg per second, in other words, the maximum increase in cavity pressure per second. While the values of the maximum increases in pressure per second have been indicated in the look-up table 23 as consecutive values from ΔP₁ mmHg per second to ΔP₂₁ mmHg per second, this is only for convenience, it will be appreciated that the actual values of ΔP mmHg per second will not increase in column 5 progressively from row 1 to row 21, but will vary as discussed above depending on the sex, age range and body max index of the subject, and also will vary depending on the cavity being insufflated.

In order to assist in an understanding of the invention, the use of the insufflator in insufflating the peritoneal cavity 3 of the subject 5 will now be described. Prior to commencing insufflating of the cavity 3 of the subject, data relating to the subject, and the target pressure to which the cavity 3 is to be insufflated, is entered into the microprocessor 20 through the touchscreen 30 by the surgeon or clinician. The data relating to the subject to be entered through the touchscreen 30 comprises the identity of the cavity of the subject to be insufflated, which in this case, is the peritoneal cavity 3, the sex of the subject, for the purpose of this example the sex of the subject is male, the age of the subject and the body mass index of the subject.

With this data entered through the touchscreen 30 into the microprocessor 20, the microprocessor 20 reads the appropriate reference value of the maximum increase in the cavity pressure per second above which the rate of insufflating the peritoneal cavity 3 of the subject should not exceed from column 5 of the look-up table 23 based on sex, age and body mass index of the subject entered through the touchscreen 30. The appropriate reference value of the maximum increase in cavity pressure per second is then stored in a memory of the microprocessor 20 for the duration of the procedure during which the peritoneal cavity of the subject is being insufflated, as is the target pressure at which the cavity 3 is to be insufflated also stored in the memory of the microprocessor 20 for the duration of the procedure during which the peritoneal cavity of the subject is being insufflated. Typically, the target pressure at which the cavity is to be insufflated for the duration of the procedure would lie in the range of 7 mmHg to 20 mmHg, and may be varied during the procedure upwardly and downwardly by the surgeon or clinician to increase or decrease the working volume in the peritoneal cavity 3 by entering the desired target pressure through the touchscreen 30.

The insufflator 1 is connected to the subject with the first conduit 18 connecting the first outlet port 15 to the insufflating gas inlet port 19 of the trocar 6, and the second conduit 25 connecting the second outlet port 24 to the insufflating gas inlet port 26 of the trocar 28. On activation of the insufflator 1 by activating an on/off button 31 on the touchscreen 30, the microprocessor 20 operates the flow controller 14 to deliver the insufflating gas to the cavity 3, and at the predefined time intervals of 10 milliseconds, the microprocessor 20 reads the signal indicative of cavity pressure from the pressure sensor 22, and each time the cavity pressure is read from the pressure sensor 22, the microprocessor 20 computes the current value of the increase of cavity pressure per second. The microprocessor 20 then compares the just computed current value of the increase in cavity pressure per second with the stored appropriate reference value of the maximum increase in cavity pressure per second. For so long as the computed values of the increase of cavity pressure per second remains below the stored appropriate reference value, the microprocessor 20 operates the flow controller 14 to continue insufflating the cavity at the current rate at which the cavity is being insufflated.

In the event of a computed value of the increase of cavity pressure per second exceeding the stored appropriate reference value of the maximum increase in cavity pressure per second, the microprocessor operates the flow controller 14 to reduce the rate of insufflating of the cavity 3, in order to reduce the increase of cavity pressure per second to or below the stored appropriate reference value. If the computed rate of increase in cavity pressure per second exceeds the stored appropriate reference value by a significant amount, the microprocessor 20 may operate the flow controller 14 to pause insufflating of the cavity before recommencing insufflating thereof.

On the microprocessor 20 determining from the signal read from the pressure sensor 22 that the cavity pressure is at the selected target pressure, the microprocessor 20 operates the flow controller 14 to reduce the flow rate of the insufflating gas to the cavity to a rate just sufficient to maintain the cavity pressure at the selected target pressure. The microprocessor 20 then continues to operate the insufflator to maintain the cavity pressure at the selected target pressure until the procedure has been completed or until a new target pressure is selected and entered through the touchscreen 30.

The microprocessor 20 is programmed to continue to read the signal from the pressure sensor 22 at the predefined time intervals of 10 milliseconds during insufflating of the cavity, and on each reading of the signal from the pressure sensor 22, the microprocessor 20 computes the increase in cavity pressure per second from the previous reading. Each computed increase in cavity pressure per second is compared with the stored appropriate reference value of the maximum increase in cavity pressure per second, and if as a result in an increase in the selected target pressure, or leakage of insufflating gas from the cavity, the computed increase in the cavity pressure per second exceeds the stored appropriate reference value, the microprocessor 20 operates the flow controller 14 to reduce the flow rate of insufflating gas to the cavity or to pause insufflating of the cavity until the increase in the cavity pressure per second is reduced to or below the stored appropriate reference value of the maximum increase in cavity pressure per second, as described above.

It is envisaged that provision may be made in the insufflator 1 for incrementally increasing or decrementally decreasing the target pressure in the cavity by, for example, a pressure-up/pressure-down button switch, or by a pair of foot pedal operated switches, or by any other suitable means. In the case of a pressure-up/pressure-down button switch, such a switch would be located in the housing, and would be configured, so that each time the pressure-up/pressure-down button switch is toggled upwardly to increase the target pressure, a signal would be outputted indicative of a request to increase the target pressure by one increment, which, may, for example, be an incremental increase in pressure of 0.5 mmHg or an incremental increase in pressure of 1 mmHg, or any other suitable size increment. Each time the pressure-up/pressure-down button switch is toggled downwardly, a signal would be outputted indicative of a request for a decrease in the target pressure by one decrement, which may, for example, also be a decrement of 0.5 mmHg or a decrement of 1 mmHg, or any other suitable size decrement. The microprocessor 20 would be programmed to read the signals from the pressure-up/pressure-down button switch, and on receiving each signal, indicative of a request for an increment increase in the target pressure or a decremental decrease in the target pressure, the microprocessor 20 would operate the flow controller 14 to either increase or decrease, as the case may be, the rate of flow of insufflating gas to the cavity to increase or decrease the cavity pressure to the new target pressure.

Additionally, it is envisaged that the pressure-up/pressure-down button switch may be configured so that when the pressure-up/pressure-down button switch is toggled upwardly and held in the upward position, the pressure-up/pressure-down button switch would sequentially produce a plurality of signals for so long as it is held in the upward position, each signal being indicative of a request for an increase in the target pressure by on increment. Each time the pressure-up/pressure-down button switch is toggled downwardly and held in the downward position, the pressure-up/pressure-down button switch would sequentially produce a plurality of signals for so long as it is held in the downward position, each signal being indicative of a request for a decrease in the target pressure by one decrement. The signal processor 20 would count the number of signals produced by the pressure-up/pressure-down button switch, and operate the flow controller 14 to increase or decrease, as the case may be, the cavity pressure by the appropriate number of incremental increases or decremental decreases corresponding to the number of signals produced by the pressure-up/pressure-down button switch.

In the case of a pair of foot pedal switches being provided, one of the foot pedal switches would be configured to produce a signal indicative of an incremental increase in target pressure, and the other foot pedal operated switch would be configured to produce a signal indicative of a decremental decrease in the target pressure. The foot pedal operated switches typically, would be configured so that each time one of the foot pedal operated switches is operated into the closed state, a signal indicative of a request for an incremental increase in the target pressure by one increment would be produced, and each time the other one of the foot operated pedal switches would be operated into the closed state, a signal indicative of a request for a decremental decrease in the target pressure by one decrement would be produced. Additionally, if the respective foot pedal operated switches were held down for a continuous period, the corresponding foot pedal operated switch would sequentially produce a plurality of signals each indicative of a request for either an increase, or a decrease in the target pressure by one increment or one decrement, as the case may be, and the signal processor would count the number of signals produced by the relevant foot pedal operated switch, and the microprocessor 20 would operate the flow controller 14 to increase or decrease as the case may be, the flow of insufflating gas to the cavity to in turn increase or decrease the target pressure by the appropriate number of incremental pressure increases or the appropriate number of decremental decreases.

On each request for an increase in the target pressure through either the pressure-up/pressure-down button switch or through the foot pedal operated switches, the microprocessor 20 would continue to monitor the signal from the pressure sensor 22 at the predefined time intervals of 10 milliseconds, and on each reading of the signal from the pressure sensor 22, the microprocessor 20 would compute the increase in cavity pressure per second since the previously read signal from the pressure sensor 22.

Each computed increase in the cavity pressure per second would be compared with the stored appropriate reference value of the maximum increase in cavity pressure per second, and if the computed cavity pressure per second exceeded the stored appropriate reference value, the microprocessor 20 would operate the flow controller 14 to reduce or pause insufflating of the cavity 3 until the current increase in the cavity pressure per second at which the cavity is being insufflated, fell to or below the stored appropriate reference value of the maximum increase in cavity pressure per second, as already described.

It is also envisaged that the microprocessor 20 may be programmed to allow the rate at which the cavity is being insufflated to exceed the stored appropriate reference value of the maximum increase in cavity pressure per second during initial insufflating of the cavity, for example, depending on the cavity until the cavity had been insufflated to a predefined volume or a predefined pressure. The predefined volume or the predefined pressure may, for example, be a volume or a pressure equal to a predefined proportion of the maximum safe volume or the maximum safe pressure to which the cavity may be insufflated. The predefined proportion of the maximum safe volume or the maximum safe pressure to which the cavity may be insufflated may, for example, lie in the range of 40% to 80%, and more typically, would lie in the range of 50% to 75% of the maximum safe volume or the maximum safe pressure to which the cavity may be insufflated.

It is envisaged that in cases where the microprocessor 20 is programmed to allow the rate of insufflating of the cavity to exceed the stored appropriate reference value of the maximum increase in cavity pressure per second until the cavity had been insufflated to the predefined volume or the predefined pressure, the relevant predefined volume or the relevant predefined pressure would be stored in the look-up table for each type of cavity, for each type of subject, namely, male and female subjects, subjects of the different age ranges and subjects of the different body mass index ranges, and would be cross-referenced therewith in the look-up table.

During insufflating of the cavity, once the cavity volume or cavity pressure reached the stored predefined volume or the predefined pressure corresponding to the subject and cavity being insufflated, if the rate at which the cavity is being insufflated exceeds the stored appropriate reference value of the maximum increase in cavity pressure per second, the microprocessor 20 would operate the flow controller 14 to reduce or pause insufflating of the cavity until the increase in the cavity pressure per second fell below the stored appropriate reference value of the maximum increase in cavity pressure per second.

It is also envisaged that in some embodiments of the invention as well as storing the reference values of the maximum increase in cavity pressure per second, values of rates at which insufflating of the cavity should be progressively reduced as the cavity is being insufflated to the target pressure may also be stored and cross-referenced with the cavity or the type of cavity, the age or age ranges of subjects, the body mass indices or the body mass index ranges of subjects and the sex of subjects. The values of the rates at which the increase in pressure per second should be progressively reduced may be stored to be applied from the commencement of insufflating of the cavity, or from the cavity pressure reaching the predefined pressure or the volume of the cavity reaching the predefined volume or being above the predefined pressure or the predefined volume. The microprocessor would be programmed at the appropriate time to commence progressive reduction of the increase in cavity pressure per second by the appropriate stored value of the rate thereof.

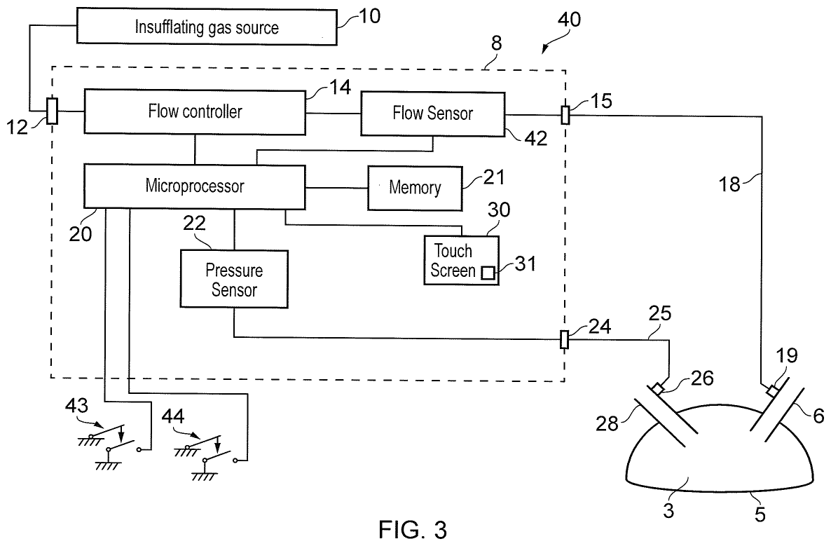

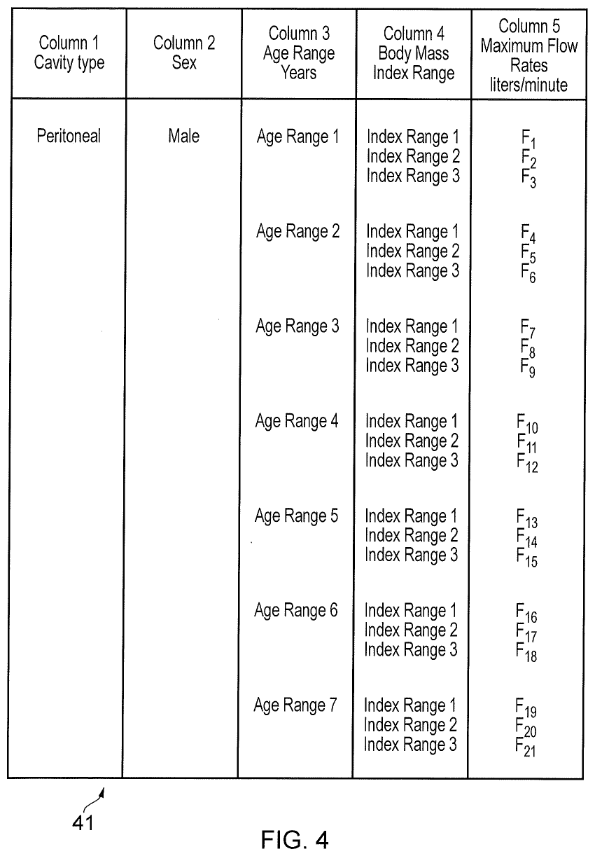

Referring now to FIGS. 3 and 4 there is illustrated an insufflator according to another embodiment of the invention indicated generally by the reference numeral 40 for insufflating a cavity in the body of a human or animal subject. The insufflator 40 is substantially similar to the insufflator 1 and similar components are identified by the same reference numerals. In the embodiment of the invention the insufflator 40 will be described for insufflating a peritoneal cavity 3, similar to that described with reference to FIG. 1, in the body 5 of a human subject. The main difference between the insufflator 40 and the insufflator 1 is in the manner in which the microprocessor 20 is programmed for determining the rate of insufflating of the cavity 3. In this embodiment of the invention the microprocessor 20 is programmed to determine the rate of insufflating of the cavity 3 as a function of the flow rate of the insufflating gas being delivered to the cavity 3, and the microprocessor 20 is programmed to determine the flow rate of insufflating gas to the cavity 3, as will be described below.

Look-up tables in a format substantially similar to the look-up table 23 of FIG. 2 are stored in the memory 21. A part of the look-up table 41 is illustrated in FIG. 4 for the peritoneal cavity of males of respective different age ranges and respective different body mass index ranges. Columns 1 to 4 of the look-up table 41 are substantially similar to columns 1 to 4 of the look-up table 23. However, in column 5 of the look-up table 41, the reference values of the maximum rates of insufflating the cavities are stored as reference values of maximum flow rates of insufflating gas to the peritoneal cavity 3, above which the flow rates of insufflating gas to the cavities should not exceed in order to avoid any risk of bradycardia, hypotension or cardiac arrest in the subject. In this case, the maximum flow rates are stored in column 5 as litres per minute, and are represented as F₁ litres per minute to F₂₁ litres per minute.

Although, the look-up table 41 is only provided for the peritoneal cavity 3 of male subjects, similar type look-up tables are provided for the peritoneal cavity of female subjects of different age and body mass index ranges, as well as for other cavities of male and female subjects of different age and body mass index ranges.

In the look-up table 41, seven age ranges similar to the seven age ranges of the look-up table 23 are provided for male subjects and three body mass index ranges similar to the three body mass index ranges of the look-up table 23 are provided for male subjects.

A monitoring means in this case comprising a flow sensor 42 is located in the housing 8 between the flow controller 14 and the outlet port 15. The flow sensor 42 monitors the flow of insufflating gas to the cavity 3 and produces a signal indicative of the flow rate of insufflating gas being delivered to the cavity 3 in litres per minute. The microprocessor 20 is programmed to read the signal from the flow sensor 42 at the predefined time intervals of 10 milliseconds, and to compare the current value of the flow rate at which insufflating gas is being delivered to the cavity with the stored appropriate one of the reference values of the maximum flow rate of insufflating gas from column 5 of the look-up table 41. If the computed flow rate at which insufflating gas is being delivered to the cavity exceeds the stored appropriate reference value thereof, the microprocessor is programmed to operate the flow controller 14 to reduce the flow rate of insufflating gas to the cavity or to pause the delivery of insufflating gas to the cavity until the flow rate of insufflating gas being delivered to the cavity falls to or below the stored appropriate reference value thereof.

In this embodiment of the invention a pair of foot pedal operated switches are provided, namely, a first foot pedal operated switch 43, and a second foot pedal operated switch 44. The first and second foot pedal operated switches 43 and 44 are located externally of the insufflator 40 and are hardwire connected to the microprocessor 20, and are provided to produce signals to the microprocessor 20 indicative of an increase or a decrease in the target pressure at which the cavity 3 is to be insufflated. However, in some embodiments of the invention it is envisaged that the first and second foot pedal operated switches 43 and 44 may communicate wirelessly with the insufflator 40, and in which case, each first and second foot pedal operated switches 43 and 44 would be provided with a radio transmitter, and a radio receiver tuned to the radio transmitter would be located in the housing 8 of the insufflator 40. The microprocessor 20 would read signals from the wireless receiver received from the first and second foot pedal operated switches 43 and 44.

In this embodiment of the invention the first foot pedal operated switch 43 is provided for increasing the target pressure, while the second foot pedal operated switch 44 is provided for decreasing the target pressure. The first foot pedal operated switch 43 is configured so that each time the foot pedal operated switch 43 is operated from the open-circuit state into the closed-circuit state thereof, the foot pedal operated switch 43 is configured to produce a single signal indicative of a request for an incremental increase in the target pressure by a single pressure increment, in this embodiment of the invention a pressure increment in the range of 0.5 mmHg to 1 mmHg. The second foot pedal operated switch 44 is configured such that each time the second foot pedal operated switch 44 is operated from the open-circuit state into the closed-circuit state, a signal is produced indicative of a request for a decremental decrease in the target pressure by a single pressure decrement, in this embodiment of the invention a decrement in the range of 0.5 mmHg to 1 mmHg. Additionally, the first and second foot pedal operated switches 43 and 44 are configured, so that when held in the closed-circuit state thereof the corresponding one of the first and second foot pedal operated switches 43 or 44 produce a plurality of sequential signals, each signal being indicative of a request for either an incremental increase in the target pressure by a single pressure increment, or a decremental decrease in the target pressure by a single pressure decrement, as the case may be.

The microprocessor 20 is programmed to read the signals produced by the first and second foot pedal operated switches 43 and 44. On the first foot pedal operated switch 43 being operated to produce a single signal indicative of an increase in the target pressure by one increment, the microprocessor 20 operates the flow controller 14 to increase the flow of insufflating gas to the peritoneal cavity 3 in order to increase the target pressure at which the cavity is currently being insufflated by one pressure increment. If the first foot pedal operated switch 43 were held in the closed-circuit state to produce a number of sequential signals each indicative of an increase in the target pressure by one pressure increment, the microprocessor 20 is programmed to count the number of sequential signals produced by the first foot pedal operated switch 43, and to then operate the flow controller 14 to increase the flow of insufflating gas to the peritoneal cavity 3 in order to increase the target pressure at which the peritoneal cavity 3 is being insufflated by the appropriate number of pressure increments.

On the foot pedal operated switch 44 being operated to produce a single signal, the microprocessor 20 operates the flow controller 14 to reduce the delivery of insufflating gas to the cavity 3 to decrease the target pressure at which the cavity is being insufflated by a single pressure decrement. On the second foot pedal operated switch 44 being operated to produce a plurality of sequential signals, the microprocessor 20 counts the number of sequential signals produced by the second foot pedal operated switch 44 and operates the flow controller to either reduce or pause delivery of insufflating gas to the cavity until the target pressure at which the cavity is being currently insufflated has been reduced by the appropriate number of pressure decrements to the new target pressure.

In use, as with the insufflator 1, the surgeon or clinician enters particulars of the cavity to be insufflated, the sex of the subject, the age of the subject and the body mass index of the subject through the touchscreen 30. The surgeon or clinician also enters the target pressure at which the peritoneal cavity 3 is to be insufflated during insufflating thereof. With the insufflator 40 connected to the cavity with the first conduit 18 connected to the insufflating gas inlet port 19 of the trocar 6, and the second conduit 25 connected to the insufflating gas inlet port 26 of the trocar 28, the surgeon or clinician activates the insufflator 40 to insufflate the peritoneal cavity 3 of the subject to the selected target pressure. The microprocessor 20 determines the appropriate reference value of the maximum flow rate of insufflating gas to the cavity from the look-up table 41 based on the sex, age and body mass index of the subject entered through the touch screen 30, and stores the determined appropriate reference value of the maximum flow rate of insufflating gas to the cavity in its memory as well as the target pressure.

The microprocessor 20 then reads the signal from the flow sensor 42 at the predefined time intervals of 10 milliseconds, and compares the current flow rate at which insufflating gas is being delivered to the cavity with the stored appropriate reference value of the maximum flow rate of insufflating gas to the cavity stored in the microprocessor 20 from the table 41.

If the current flow rate of insufflating gas to the cavity is less than the stored appropriate reference value thereof, the microprocessor 20 continues to operate the flow controller 14 to increase the cavity pressure to the target pressure. On the cavity pressure reaching the target pressure, the microprocessor 20 operates the flow controller 14 to maintain the cavity pressure at the target pressure. If however the current flow rate of insufflating gas to the cavity 3 exceeds the stored appropriate reference value, the microprocessor 20 operates the flow controller 14 to reduce the flow rate of insufflating gas to the cavity, or to pause the flow of insufflating gas to the cavity until the flow rate of insufflating gas being delivered to the cavity falls to or below the stored appropriate reference value thereof, before recommencing flow of insufflating gas to the cavity.

If during insufflating of the cavity 3, the surgeon or clinician wishes to increase or decrease the target pressure at which the cavity is to be insufflated, the surgeon or clinician enters the new target pressure through the touchscreen 30. The microprocessor 20 on detecting the newly entered target pressure from the touchscreen 30 operates the flow controller 14 to increase or decrease or pause the flow of insufflating gas to the cavity in order to increase or decrease the cavity pressure to the newly selected target pressure.

Additionally, should the surgeon or clinician wish to increase or decrease the target pressure during insufflating of the cavity 3, the surgeon or clinician may operate the appropriate one of the first or second foot pedal operated switches 43 or 44, namely, the first foot pedal operated switch 43 if the target pressure is to be increased and the second foot pedal operated switch 44 if the target pressure is to be decreased. The microprocessor 20 reads the signals from the first and second foot pedal operated switches 43 and 44, and counts the number of signals produced by either one of the first or second foot pedal operated switches 43 and 44. The microprocessor 20 then controls the flow controller 14 to either increase or decrease or pause the flow of insufflating gas to the cavity in order to increase or decrease, as the case may be, the cavity pressure to the new target pressure.

During increasing of the cavity pressure to a new target pressure in response to an increase in the target pressure entered through the touch screen 30, or in response to the signal or signals read from the first foot pedal operated switch 43, or in the event of an increase in leakage of insufflating gas from the cavity, if the current value of the flow rate of insufflating gas to the cavity 3 exceeds the stored appropriate reference value from column 5 of the look-up table 41, the microprocessor 20 operates the flow controller 14 to reduce or pause the flow of insufflating gas to the cavity 3 until the current value of the flow rate of insufflating gas to the cavity 3 has been reduced to or below the stored appropriate reference value.

Otherwise, the insufflator 40 and its operation is similar to the insufflator 1 and its operation.

It will also be appreciated that the microprocessor may be programmed to only take action to reduce the flow rate of insufflating gas to the cavity or to pause the flow of insufflating gas to the cavity in the event of the current flow rate of insufflating gas to the cavity exceeding the stored appropriate reference value until after the volume of the cavity or the cavity pressure has reached the predefined volume or the predefined pressure. In which case the look-up table would include values of the predefined volumes or the predefined pressures corresponding to the respective reference values of the maximum flow rates of insufflating gas to the cavity.

Additionally, if the look-up table includes values of the rates at which the reference values of the maximum flow rates of insufflating gas to the cavity should be progressively reduced either from the commencement of insufflating of the cavity or from the cavity pressure or the cavity volume reaching the predefined pressure or the predefined volume, the microprocessor would be programmed to appropriately progressively reduce the stored appropriate reference value of the maximum flow rate of insufflating gas to the cavity as the cavity pressure or the cavity volume increases either from the commencement of insufflating of the cavity or from the cavity pressure reaching the predefined pressure or the cavity volume reaching the predefined cavity volume.

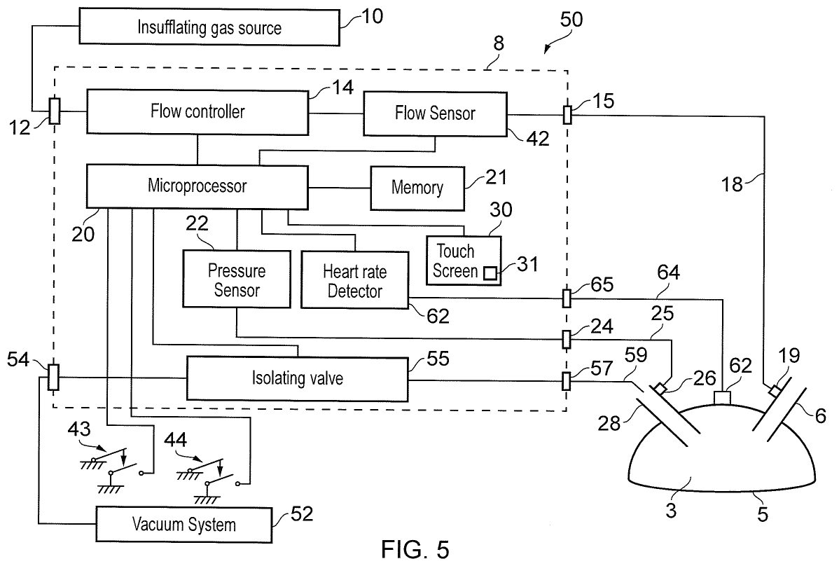

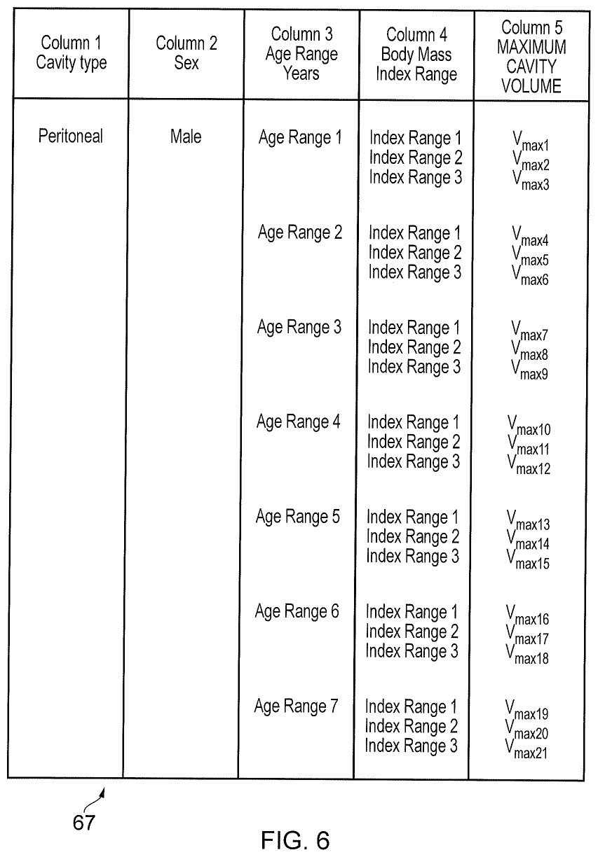

Referring now to FIGS. 5 and 6 there is illustrated an insufflator according to another embodiment of the invention indicated generally by the reference numeral 50 for insufflating a cavity 3 in the body 5 of a human subject. The insufflator 50 is substantially similar to the insufflator 1, and similar components are identified by the same reference numerals.

The main differences between the insufflator 50 and the insufflator 1 is that as well as monitoring the rate of increase in cavity pressure per unit time and operating the flow controller 14 for controlling the flow rate of insufflating gas being delivered to the cavity 3 in order to avoid the increase in cavity pressure per unit time exceeding the relevant reference value of the maximum increase in cavity pressure per unit time, the insufflator 50 is also adapted to monitor the volume of the cavity 3 during insufflating thereof in order to avoid the cavity 3 exceeding a predefined maximum volume. Additionally, the insufflator 50 is adapted to monitor a characteristic indicative of the performance of the heart of the subject, and to control insufflating of the cavity 3 in order to avoid the characteristic indicative of the performance of the heart of the subject falling below a predefined minimum value thereof, and/or exceeding a predefined maximum volume thereof. In this embodiment of the invention the monitored characteristic indicative of the performance of the heart of the subject is the heart rate of the subject, and predefined minimum and maximum values of the heart rate of a subject are stored, as will be described below.

The insufflator 50 is also adapted for monitoring the flow of insufflating gas being delivered to the cavity 3, and comprises a flow sensor, similar to the flow sensor 42 of the insufflator 40 described with reference to FIGS. 3 and 4. The flow sensor 42 is located between the flow controller 14 and the first outlet port 15 for monitoring the flow of insufflating gas delivered to the cavity 3. The microprocessor 20, as will be described below, reads signals from the flow sensor 42 for determining the cumulative value of insufflating gas delivered to the cavity 3 from the commencement of insufflating thereof. The insufflator 50 is also adapted for applying a vacuum to the cavity 3 of a subject being insufflated for withdrawing insufflating gas therefrom to depressurise the cavity 3 in response to the heart rate of the subject falling below the predefined heart rate value or exceeding the predefined maximum heart rate value, or in the event of the volume of the cavity exceeding the predefined maximum volume.

Additionally, the insufflator 50 comprises a pair of foot pedal operated switches 43 and 44, which are similar to the foot pedal operated switches 42 and 43 of the insufflator 40 described with reference to FIGS. 3 and 4, and are provided for increasing and decreasing the target pressure, in a similar manner as described with reference to the insufflator 40.

The insufflator 50 is adapted for connecting to an external vacuum system, in this embodiment of the invention an external vacuum system 52 of the type typically available in an operating theatre of a hospital in which the insufflator 50 would be used. An inlet vacuum port 54 in the housing 8 is provided for coupling the insufflator 50 to the vacuum system 52. An isolating valve 55 located in the housing 8 is connected between the inlet vacuum port 54 and an outlet vacuum port 57 in the housing 8 through which a vacuum is applied to the cavity 3. The isolating valve 55 is operated under the control of the microprocessor 20 from an isolating state isolating the cavity 3 from the vacuum source 52 to an open state for selectively applying vacuum to the cavity 3. A vacuum line comprising a third conduit 59 is connected to the outlet vacuum port 57, and extends into the cavity 3 through an instrument channel of the trocar 28 for applying the vacuum to the cavity 3. However, in some embodiments of the invention it is envisaged that the third conduit 59 may be entered into the cavity 3 through any other suitable means, for example, through a separate trocar, or the third conduit 59 may be connected to an insufflating inlet port of a trocar or to a Veress needle extending into the peritoneal cavity 3, or by any other means.

A heart rate detector 60 located in the housing 8 is configured to receive signals from a heart rate monitor 62 attached to the body 5 of the subject. The heart rate monitor may be any suitable heart rate monitor, for example, one or more heart rate sensors attached to the chest of the subject or any other suitable heart rate monitor capable of producing a signal indicative of the heart beat of the heart of the subject. The heart rate detector in this embodiment of the invention is hardwired to the heart rate monitor 62 through a cable 64 connected to the heart rate detector 60 through a releasable connector 65 located in the housing 8. A signal indicative of the heart rate of the subject is transmitted from the heart rate monitor 62 to the heart rate detector 60, which produces a signal readable by the microprocessor 20 indicative of the heart rate of the subject. The signal indicative of the heart rate of the subject produced by the heart rate detector 60 is read by the microprocessor 20 at the predefined time intervals of 10 milliseconds.

Two heart rate values are stored in the memory 21, one of which heart rate values is a predefined maximum heart rate value, above which the heart rate of the subject ideally should not exceed during insufflating of the cavity 3, and the other heart rate value is a predefined minimum heart rate value, below which the heart rate of the subject ideally should not fall during insufflating of the peritoneal cavity 3. Typically, the predefined minimum heart rate value is approximately 60 beats per second. The predefined maximum heart rate value typically is selected and entered through the touch screen 30 by the surgeon or clinician.

The microprocessor 20 is programmed each time the signal from the heart rate detector 60 is read to compare the read value of the heart rate of the subject with the stored predefined maximum heart rate value and the stored predefined minimum heart rate value, and in the event of the read heart rate of the subject exceeding the predefined maximum heart rate value or falling below the predefined minimum heart rate value, the microprocessor 20 is programmed to operate the flow controller to pause delivery of insufflating gas to the cavity 3. The microprocessor 20 is programmed to continue reading the signal of the heart rate of the subject at the predefined time intervals from the heart rate detector 60, and if after a first predefined time period of, for example, 30 seconds, the heart rate of the subject has not risen to or above the predefined minimum heart rate value, or if after a second predefined time period also of approximately 30 seconds the heart rate of the subject has not fallen to or below the predefined maximum heart rate value, as the case may be, the microprocessor 20 is programmed to operate the isolating valve 55 from the isolating state to the open state for applying the vacuum to the cavity 3 to depressurise the peritoneal cavity 3.

Turning now to the operation of the insufflator 50 for monitoring the volume of the cavity 3 of the subject and for controlling the insufflating of the cavity 3 to avoid the volume of the cavity 3 exceeding the predefined maximum volume, in this embodiment of the invention the microprocessor 20 is programmed to operate the flow controller 14 and/or the isolating valve 55 in order to avoid the volume of the peritoneal cavity 3 being insufflated beyond the predefined maximum volume. The predefined maximum volume of the cavity 3 of the subject, beyond which the cavity 3 of the subject ideally should not be insufflated is obtained from a look-up table 67 also stored in the memory 21 and illustrated in FIG. 6. The layout of the look-up table 67 is substantially similar to that of the look-up table 23. The predefined maximum volume beyond which the cavity in the body of a human subject should not be insufflated depends on the cavity itself, as well as the sex of the subject, the body mass index of the subject, and in particular, the predefined maximum volume depends on the age of the subject.

Accordingly, in this embodiment of the invention the look-up table 67 sets out a plurality of predefined maximum volume values for different cavities in the bodies of male and female subjects, as well as male and female subjects of different age ranges and different body mass index ranges. However, only a part of the look-up table 67 is illustrated in FIG. 6, which sets out the predefined maximum volume values for the peritoneal cavity of male subjects of seven age ranges, similar to the seven age ranges of the look-up table 23 of the insufflator 1, and three body mass index ranges for each of the seven age ranges are also provided, similar to the three body mass index ranges of the look-up table 23. Column 1 of the look-up table 67 includes particulars of the cavity. Column 2 of the look-up table 67 includes particulars of the sex of the subject. Column 3 includes the age ranges of the subject, while column 4 includes the body mass index ranges of the subject. Column 5 of the look-up table 67 includes the predefined maximum volume values for the cavity of the male subjects of the different age ranges and the different body mass index ranges.

The microprocessor 20 is programmed to monitor the volume of the cavity 3 being insufflated, and on the volume of the cavity 3 exceeding the stored appropriate predefined maximum volume value read from the look-up table 67, the microprocessor 20 is programmed to either operate the flow controller 14 to pause or terminate insufflating of the cavity, and/or to operate the isolating valve 55 from the isolating state to the open state to apply a vacuum to the cavity 3 to depressurise the cavity 3, and in turn, to reduce the volume thereof. If the current volume of the cavity is only marginally above the stored appropriate predefined maximum volume value read from the look-up table 67, the microprocessor 20 is programmed to operate the flow controller 14 to pause insufflating of the cavity 3 until the volume of the cavity 3 falls to or below the stored appropriate predefined maximum volume value read from the look-up table 67. On the other hand, if the volume of the cavity 3 is significantly greater than the stored appropriate predefined maximum volume value read from the look-up table 67, the microprocessor 20 is programmed to operate the flow controller 14 to pause insufflating of the cavity 3 and to operate the isolating valve 55 into the open state to apply a vacuum to the cavity 3 to in turn reduce the volume of the cavity 3 until the volume of the cavity 3 falls to or below the stored appropriate predefined maximum volume value read from the look-up table 67.

The microprocessor 20 is programmed to determine the volume of the cavity based on the total volume of insufflating gas delivered to the cavity 3, less the volume of insufflating gas leaking from the cavity 3. The microprocessor 20 reads the signal from the flow sensor 42 indicative of the flow rate of insufflating gas delivered to the cavity 3 at the predefined time intervals of 10 milliseconds from the commencement of insufflating of the cavity, and on each reading of the signal computes and stores the cumulative volume of insufflating gas delivered to the cavity from the commencement of insufflating the cavity. The microprocessor 20 is programmed to compute the rate of insufflating gas leaking from the cavity 3 by determining the flow rate of the insufflating gas to the cavity in order to maintain the cavity pressure constant. The microprocessor 20 then determines the current volume of the cavity 3 from the current cumulative volume of insufflating gas delivered to the cavity corrected for cavity pressure less the volume of insufflating gas which leaked from the cavity from the commencement of insufflating thereof.

In use, the insufflator 50 is connected to the insufflating gas source 10 through the inlet port 12, and is connected to the vacuum system 52 through the inlet vacuum port 54. The insufflator is connected to the cavity 3 of the subject with the first conduit 18 connecting the first outlet port 15 to the insufflating gas inlet port 19 of the trocar 6, and the pressure sensor 22 connected to the insufflating gas inlet port 26 of the trocar 28 by the second conduit 25, and with the third conduit 59 connected to the vacuum outlet port 57 and extending into the cavity 3 through the instrument channel of the trocar 28. The heart rate monitor 62 is connected to the heart rate detector 60 through the connector 65. With the insufflator 50 so connected, the insufflator 50 is ready for use.

The surgeon or clinician enters the type of the cavity of the subject being insufflated, in this embodiment of the invention the peritoneal cavity 3, the sex, the age and the body mass index of the subject into the microprocessor 20 through the touchscreen 30. The surgeon or clinician also enters the target pressure to which the cavity is to be insufflated during insufflating thereof.

With the data of the subject and the target pressure to which the cavity is to be insufflated entered into the microprocessor 20, the surgeon or clinician then activates the insufflator 50 by operating the on/off button switch 31 on the touchscreen 30. The microprocessor 20 reads the reference value of the maximum increase in cavity pressure per second from the look-up table 23 stored in the memory 21 appropriate for the peritoneal cavity 3 of the male subject of the age and body mass index entered through the touch screen 30, and stores the appropriate reference value of the maximum increase in cavity pressure per second in the microprocessor 20. The microprocessor 20 also reads the value of the predefined maximum volume from the look-up table 67 stored in the memory 21 appropriate to the peritoneal cavity 3 of the male subject of the age and body mass index entered through the touch screen 30, and stores the appropriate predefined maximum volume value in the memory of the microprocessor 20. The predefined maximum and minimum heart rate values are read by the microprocessor 20 from the memory 21 and stored in the memory of the microprocessor 20. The microprocessor 20 then operates the flow controller 14 to commence insufflating of the cavity 3.

As the cavity 3 is being insufflated, the microprocessor 20 reads the signals from the pressure sensor 22, the flow sensor 42, and from the heart rate detector 60, as well as from the touch screen 30 and from the first and second foot pedal operated switches 43 and 44 at the predefined time intervals of 10 milliseconds. As each signal is read from the pressure sensor 22, the microprocessor 20 computes the current increase in the cavity pressure per second and compares the computed value of the increase in cavity pressure per second with the stored appropriate reference value of the maximum increase in cavity pressure per second. If the computed value of the increase in cavity pressure per second is less than the stored appropriate reference value thereof, the microprocessor 20 operates the flow controller to continue delivering insufflating gas to the cavity until the cavity pressure reaches the target pressure, and at which stage, the microprocessor 20 operates the flow controller to deliver insufflating gas to the cavity 3 in order to maintain the cavity pressure at the target pressure.

On the other hand, if the computed increase in the cavity pressure per second exceeds the stored appropriate reference value thereof, the microprocessor 20 operates the flow controller 14 to reduce the rate of insufflating of the cavity or to pause insufflating of the cavity until the increase in the cavity pressure per second falls to or below the stored appropriate reference value. At that stage, the microprocessor 20 operates the flow controller 14 to return and maintain the cavity pressure at the target pressure.

On reading the signal from the flow controller at the end of each predefined time interval, the microprocessor 20 computes the cumulative volume of insufflating gas delivered to the cavity 3 from the commencement of insufflating thereof. Once the cavity pressure has reached the target pressure, the microprocessor 20 determines the rate of leakage of insufflating gas from the cavity by computing the volume of insufflating gas being delivered to the cavity to maintain the cavity pressure at the target value. Once the rate of leakage of insufflating gas from the cavity has been determined, the microprocessor 20 then determines the current volume of the cavity 3 from the current cumulative volume of insufflating gas delivered to the cavity from the commencement of insufflating of the cavity less the total volume of insufflating gas which has leaked from the cavity from the commencement of insufflating of the cavity to the current time, with the cumulative volume of the insufflating gas corrected for pressure.

At the end of each predefined time interval when the current volume of the cavity 3 has been computed by the microprocessor 20, the microprocessor 20 compares the current volume of the cavity 3 with the stored appropriate predefined maximum volume value. If the current computed volume of the cavity is less than the stored appropriate predefined maximum volume value, the microprocessor 20 operates the flow controller 14 to continue delivering insufflating gas to the cavity in order to maintain the cavity pressure at the target pressure.

If however, the current computed volume of the cavity exceeds the stored appropriate predefined maximum volume value, and only exceeds the stored appropriate predefined maximum volume value by a small amount, the microprocessor 20 operates the flow controller to pause delivery of insufflating gas to the cavity. However, on the other hand, if the current computed volume of the cavity 3 exceeds the stored appropriate predefined maximum volume value by a large amount, the microprocessor 20 operates the flow controller 14 to pause insufflating of the cavity 3 and operates the isolating valve 55 from the isolating state into the open state to apply the vacuum to the cavity 3, to in turn reduce the volume of the cavity 3 until the volume of the cavity 3 falls to or below the stored appropriate predefined maximum volume value. Once the volume of the cavity 3 has fallen to or below the stored appropriate predefined maximum volume value, the microprocessor 20 operates the isolating valve 55 into the isolating state and operates the flow controller 14 to reinstate delivery of insufflating gas to the cavity in order to return and maintain the cavity pressure at the target pressure.

As the signal of the current heart rate value of the subject is read at the end of each predefined time intervals of 10 milliseconds from the heart rate detector 60, the microprocessor 20 compares the current heart rate value of the subject with the predefined maximum and minimum heart rate values. For so long as the current heart rate value of the subject falls within the predefined maximum and minimum heart rate values, the microprocessor 20 operates the flow controller 14 to continue insufflating of the cavity to maintain the cavity pressure at the target pressure.

However, if the current heart rate value of the subject exceeds the predefined maximum heart rate value or falls below the predefined minimum heart rate value, the microprocessor 20 operates the flow controller to pause delivery of insufflating gas to the cavity 3. If after the first or second predefined time period of approximately 30 seconds, the heart rate of the subject has not risen to or above the predefined minimum heart rate value or has not fallen to or below the predefined maximum heart rate value, as the case may be, the microprocessor 20 operates the isolating valve from the isolating state to the open state for applying a vacuum to the cavity to depressurise the peritoneal cavity 3, which effectively terminates insufflating of the cavity.

Throughout insufflating of the cavity 3, the microprocessor 20 reads signals from the touch screen 30 and the signals from the first and second foot pedal operated switches 43 and 44 at the predefined time intervals of 10 milliseconds, and in the event of a change in the target pressure being entered through the touch screen 30, or a change in the target pressure being entered through either of the first or second foot pedal operated switches 43 or 44, the microprocessor 20 operates the flow controller 14 to alter the delivery of insufflating gas to the cavity 3, as already described with reference to the insufflator 1 and the insufflator 40 described with reference to FIGS. 1 and 2, and 3 and 4. If a large downward alteration of the target pressure is entered through the touch screen 30 or through the second foot pedal operated switch 44, the microprocessor 20 operates the isolating valve 55 from the isolating state to the open state to apply vacuum to the cavity 3 to reduce the cavity pressure to the new target pressure.

On completion of the procedure in the cavity 3, the isolating valve 55 is operated from the isolating state to the open state in order to evacuate the cavity 3.

While the insufflator 50 described with reference to FIGS. 5 and 6 has been described as monitoring insufflating of the cavity in order to avoid the rate of insufflating of the cavity exceeding a maximum rate of insufflating thereof, and has also been described for monitoring the volume of the cavity to avoid the volume thereof exceeding a maximum predefined volume value, and has further been described for monitoring the heart rate of a subject during insufflating of a cavity thereof in order to avoid the heart rate of the subject exceeding a predefined maximum value thereof or falling below a minimum predefined value thereof, it is envisaged that the insufflator 50 may be provided solely for monitoring the volume of the cavity in order to avoid the volume thereof exceeding a predefined maximum volume thereof. It is also envisaged that the insufflator 50 may be provided solely for monitoring the heart rate of a subject, a cavity of which is being insufflated in order to avoid the heart rate of the subject exceeding a predefined maximum value thereof or falling below a predefined minimum value thereof. Indeed, it is envisaged that an insufflator may be provided for insufflating a cavity of a subject and for monitoring the heart rate of the subject or other performance characteristic of the heart of the subject, and comparing the monitored heart rate or performance characteristic of the heart of the subject with a predefined minimum value of the heart rate or an appropriate minimum value of such other performance characteristic thereof in order to avoid the heart rate or such other performance characteristic of the heart of the subject falling below the predefined minimum value thereof, without any reference to a predefined maximum value thereof.

While the signal from the heart rate monitor is applied through an electrically conductive wire to the releasable connector of the insufflator 85, it is envisaged that in some embodiments of the invention the signal from the heart rate monitor may be transmitted wirelessly from the heart rate monitor for reception by a wireless receiver located in the housing of the insufflator. In which case, the microprocessor would be configured to read the signal indicative of the heart rate of the subject from the wireless receiver.

It is envisaged that instead of the insufflator 50 being adapted for connecting to an external vacuum system, such as the vacuum system 52, in some embodiments of the invention the insufflator 50 may comprise a vacuum pump located in or adjacent the housing 8 for providing the vacuum to evacuate the cavity of the subject, or a Venturi vacuum generator.

It will also be appreciated that in some embodiments of the invention the heart rate detector 60 may be omitted, and in which case, the microprocessor 20 would be programmed to receive signals directly from a heart rate monitor attached to the subject.

It is also envisaged that in some embodiments of the insufflators described with reference to the drawings, instead of providing a look-up table with a large number of age ranges and body mass index ranges, the number of age ranges and the number of body mass index ranges may be reduced. In some embodiments of the invention it is envisaged that the insufflators may be adapted to operate at two predefined reference values of the maximum rate of insufflating the cavity, one of which would be a maximum rate of insufflating a cavity in an adult subject, and the other of which would be a maximum rate of insufflating a cavity in a paediatric subject. It is also envisaged that in such cases, the insufflator may be adapted to include reference values of maximum rates of insufflating a single cavity only for an adult subject or for a paediatric subject, for example, a peritoneal cavity or for a number of different types of cavities.

It is also envisaged that a default reference value of a maximum rate of insufflating a cavity, for example, a peritoneal cavity or for insufflating a number of different cavities, may be stored in the electronic memory, and such a default reference value would be set at paediatric maximum rate of insufflating the relevant cavity, and if no cavity were specified for insufflating by a surgeon or a clinician, the insufflator would operate based on the default reference value.

It is also envisaged that a default predefined maximum volume value may also be stored in the electronic memory, and the insufflator would default to the default value of the predefined maximum volume value in the event of data relating to the sex, age and body mass index of the subject not being entered into the microprocessor.

In cases where the insufflator is adapted for insufflating a single type of cavity only, for example, the peritoneal cavity, it is envisaged that the insufflator may include a simple selector which would either select the insufflator to be configured for paediatric use or adult use.

It is also envisaged that instead of storing the reference values of the maximum rate of insufflating a cavity in a look-up table, the maximum rates of insufflating a cavity or different cavities may be included in a memory chip, which would be included in a tube set for connecting the insufflator to the cavity, and which would be adapted for insufflating a specific cavity, and would be adapted for insufflating the specific cavity in a specific type of subject, for example, an adult subject or a paediatric subject. For example, the tube set may be a tube set adapted for connecting the insufflator to the peritoneal cavity of a subject within a certain paediatric age range, or an adult within a certain age range, or the tube set may be a tube set for use in connecting the insufflator to a paediatric peritoneal cavity or a tube set for connecting the insufflator to an adult peritoneal cavity. The memory chip would be located in the tube set, so that when the tube set is connected to the ports 15, 24 and 64, as the case may be, the memory chip would automatically connect to an electrical connector located in the relevant port 15, 24 or 64 which in turn would be connected to the microprocessor.

It is also envisaged that instead of or in addition to including reference values in the look-up table 23 based on body mass index ranges, the reference values may also be based on the weight ranges and/or height ranges of subjects.

While the characteristic indicative of the performance of the heart of a subject has been described in the embodiment of the invention with reference to FIGS. 5 and 6, as being the heart rate of the subject, it is envisaged that any other suitable performance characteristic of the heart besides the heart rate may be monitored, and the signal processor would be responsive to that monitored characteristic in the same way as it has been described as being responsive to the heart rate of the subject. For example, it is envisaged that the characteristic indicative of the performance of the heart of the subject may be the blood pressure of the subject, and in which case the microprocessor of the insufflator would be configured to read the signal indicative of the blood pressure of the subject at the predefined time intervals or substantially continuously, and each time the signal indicative of the blood pressure of the subject is read, the microprocessor would compare the read signal indicative of the blood pressure of the subject with stored predefined maximum and/or minimum systolic blood pressure values and predefined maximum and/or minimum diastolic blood pressure values. If the read systolic or diastolic blood pressure values exceeded the stored predefined maximum systolic or diastolic blood pressure values or fell below the predefined minimum systolic or diastolic blood pressure values, the microprocessor would operate the flow controller to terminate insufflating of the cavity, and if the blood pressure of the subject failed to fall below the predefined maximum systolic and/or diastolic blood pressure values or failed to increase above the predefined minimum systolic and/or diastolic blood pressure values within the first or second predefined time period, the microprocessor would operate the insufflator to withdraw insufflating gas from the peritoneal cavity as already described with reference to the insufflator of FIGS. 5 and 6.

It is also envisaged that the insufflators described with reference to FIGS. 1 and 2, and FIGS. 3 and 4, may also be adapted to receive a signal indicative of a characteristic of the performance of the heart of a subject, and in which case, the signal processor thereof would also be responsive to the signal indicative of the characteristic of the performance of the heart of the subject for controlling the operation of the flow controllers of the insufflators.

Additionally, it is envisaged that the insufflators described with reference to FIGS. 1 to 6 may also be programmed to receive a signal indicative of the blood pressure of a subject, for example, from a blood pressure monitor attached to the subject, and the microprocessor would be adapted to read the signal indicative of the blood pressure of the subject at the predefined time intervals, and each time the signal indicative of the blood pressure of the subject is read, the microprocessor would compare the read signal indicative of the blood pressure of the subject with a stored predefined maximum blood pressure value or a stored predefined minimum blood pressure value, and if the read blood pressure value of the subject exceeded the stored maximum blood pressure value or fell below the minimum blood pressure value, the microprocessor would operate the flow controller to pause or terminate insufflating of the cavity, and if the blood pressure of the subject failed to fall to or below the stored maximum blood pressure value or failed to increase to or above the stored predefined minimum blood pressure value within either the first or second predefined time periods, the signal processor would be programmed to operate the insufflator to evacuate the cavity of the subject, by applying a vacuum to the cavity of the subject, for example, by operating an isolating valve similar to the isolating valve 55 of the insufflator of FIGS. 5 and 6 to apply vacuum to the cavity, or would operate a vacuum pump in the housing of the relevant insufflator or external thereto to apply a vacuum to the subject.

Additionally, it is envisaged that each time the signal indicative of the blood pressure is read by the microprocessor, the read signal would be compared with a stored minimum blood pressure value below which the blood pressure of the subject should not fall, and if the blood pressure of the subject fell below the stored minimum blood pressure value, the microprocessor would operate the flow controller to pause or terminate insufflating of the cavity, and if the blood pressure of the subject failed to increase above the stored minimum blood pressure value within a predefined time period, the microprocessor would operate the insufflator to apply a vacuum to the cavity of the subject as already described.

While the flow sensor 42 of the insufflator 40 has been described as producing a signal indicative of the flow rate of insufflating gas being delivered to the cavity 3, in some embodiments of the invention the flow sensor may be of the type which produces a signal indicative of the flow of insufflating gas to the cavity. In which case, each time the microprocessor reads the signal from the flow sensor 42, the microprocessor would compute the flow rate of the insufflating gas being delivered to the cavity 3, and would then compare the computed flow rate of insufflating gas to the cavity 3 with the stored selected appropriate reference value.

It will also be appreciated that while the signal processor has been described as comprising a microprocessor, any suitable signal processor, for example, a microcontroller or any other such signal processor may be used. It will of course be appreciated that any suitable interface besides a touchscreen and/or foot pedal operated switches may be provided for inputting data to the microprocessor.

While the insufflators have been described for insufflating a peritoneal cavity in a human subject, the insufflators may be used for insufflating any cavity in the body of a human or animal subject, and in which case, as discussed above, appropriate look-up tables will be provided containing the relevant maximum rate of insufflating the relevant cavities in either or both human and animal subjects.

Additionally, it will be appreciated that while the look-up tables of the insufflators have been provided for the cavities of male subjects of specific age and weight ranges, a corresponding look-up table for peritoneal cavities in female subjects of similar age ranges and weight ranges will be stored in the memory. Furthermore, it will be appreciated that in some embodiments of the invention more or less age ranges may be provided in each look-up table, and more or less body mass index ranges may be provided in the look-up table.

While the insufflating gas has been described as carbon dioxide, any other suitable insufflating gas may be used.

Needless to say, while the insufflating gas has been delivered to the cavity through an insufflating gas port of a trocar, in cases where a trocar is provided without an insufflating gas port, the conduit of the first gas line may be entered into the cavity through any other means, for example, directly through an instrument channel of a trocar.

It will also be appreciated that the cavity pressure may be monitored through any other suitable communicating means, and in some cases may be monitored through a conduit extending into the cavity through an instrument channel of a trocar. It is also envisaged that in some embodiments of the invention the pressure sensor may be located in the cavity, for example, on the outer surface of a trocar adjacent a distal end of the trocar which would be located within the cavity, and in which case, a signal indicative of the pressure in the cavity would be transmitted to the microprocessor, either wirelessly or through hardwiring. It is also envisaged that the cavity pressure may be monitored through the first conduit, and in which case, insufflating of the cavity would have to be paused each time a cavity pressure reading is being taken by the pressure sensor 22, or alternatively, the microprocessor would be programmed to determine the pressure in the cavity by using a compensating factor to compensate for the reduction in pressure along the length of the first conduit due to flow resistance therein.

While the flow sensor has been described as being a component separate from the flow controller, it is envisaged that in some embodiments of the invention the flow sensor may be incorporated into the flow controller.

It is also envisaged that when determining the rate at which the cavity is being insufflated, for example, when determining the increase in cavity pressure per unit time or when reading the flow rate of insufflating gas to the cavity from the flow sensor, a smoothing algorithm may be applied to the values. Any suitable smoothing algorithm may be applied to the read values, for example, a moving average smoothing average algorithm, or any other suitable smoothing algorithm.

1. An insufflator for insufflating a cavity in the body of a human or animal subject, the insufflator comprising:
a flow controller for controlling delivery of insufflating gas to the cavity,
an electronic memory storing a reference value of a maximum rate of insufflating the cavity,
a monitoring means for monitoring insufflating of the cavity and for producing a signal indicative of the insufflating of the cavity, and
a signal processor, the signal processor being programmed
to read the signal produced by the monitoring means indicative of the insufflating of the in cavity,
to determine the rate at which the cavity is being insufflated from the signal read from the monitoring means,
to compare the determined rate at which the cavity is being insufflated with the stored reference value, and
to control the flow controller to prevent the rate at which the cavity is being insufflated exceeding the stored reference value.

2. An insufflator as claimed in claim 1 in which the signal processor is programmed to control the flow controller to reduce the flow rate at which insufflating gas is being delivered to the cavity or to pause delivery of insufflating gas to the cavity if the determined rate at which the cavity is being insufflated exceeds the stored reference value of the maximum rate of insufflating the cavity.

3. An insufflator as claimed in claim 1 in which the reference value of the maximum rate of insufflating of the cavity stored in the electronic memory is stored as a function of the pressure in the cavity (cavity pressure), and preferably, as a function of one or more of the type of cavity, the sex of the subject, the age of the subject, the weight of the subject, the height of the subject, and the body mass index of the subject, and preferably, the monitoring means for monitoring insufflating of the cavity comprises a pressure sensor configured to monitor the cavity pressure and to produce a signal indicative of the cavity pressure, and the signal processor is programmed to determine the rate at which the cavity is being insufflated as a function of the cavity pressure from the signal produced by the pressure sensor, and preferably, the reference value of the maximum rate of insufflating the cavity stored in the electronic memory is stored as the maximum increase in cavity pressure per unit time, and advantageously, the signal processor is programmed to determine the increase in cavity pressure per unit time from the signal read from the pressure sensor.

4. An insufflator as claimed in claim 1 in which the reference value of the maximum rate of insufflating the cavity is stored in the electronic memory as a function of the flow of insufflating gas delivered to the cavity, and preferably, as a function of at least one of the type of cavity, the sex of the subject, the age of the subject, the weight of the subject, the height of the subject, and the body mass index of the subject, and preferably, the monitoring means comprises a flow sensor for monitoring flow of insufflating gas to the cavity, and for producing a signal indicative of the flow of insufflating gas to the cavity, and the signal processor is programmed to determine the rate at which the cavity is being insufflated as a function of the flow of insufflating gas to the cavity from the signal produced by the flow sensor, and preferably, the reference value of the maximum rate of insufflating the cavity stored in the electronic memory is stored as the maximum flow rate of insufflating gas to the cavity, and the signal processor is programmed to determine the rate of flow of insufflating gas to the cavity from the signal read from the flow sensor.

5. An insufflator as claimed in claim 1 in which a plurality of reference values of maximum rates of insufflating the cavity are stored in the electronic memory for respective subjects of at least one or more of different sexes, different ages, different weights, different heights, and different body mass indices, and preferably, the signal processor is programmed to determine the reference value of the maximum rate of insufflating the cavity from the electronic memory appropriate to the cavity in response to the data indicative of the one or more of the sex, the age, the weight, the height, and the body mass index of the subject inputted to the signal processor, and preferably, a plurality of the reference values are stored in the electronic memory for each one of a plurality of different types of cavities of the subject of the one or more different sexes, different ages, different weights, different heights, and different body mass indices.

6. An insufflator as claimed in claim 1 in which a predefined minimum value of a predefined characteristic indicative of the performance of a heart of a subject is stored in the electronic memory and the signal processor is programmed to read a signal indicative of a characteristic of the performance of the heart of the subject corresponding to the predefined characteristic indicative of the performance of the heart of a subject, the predefined minimum value of which is stored in the electronic memory, and on the value of the signal read by the signal processor indicative of the characteristic of the performance of the heart of the subject falling below the stored predefined minimum value thereof, the signal processor is programmed to operate the flow controller to reduce or pause delivery of insufflating gas to the cavity, and/or to operate a vacuum applying means to apply a vacuum to the cavity for reducing the cavity pressure.

7. An insufflator as claimed in claim 6 in which the signal processor is programmed to operate the flow controller and/or the vacuum applying means to reduce the cavity pressure until the signal read by the signal processor indicative of the characteristic of the performance of the heart of the subject commences to rise above the stored predefined minimum value, and preferably, the predefined minimum value of the predefined characteristic indicative of the performance of the heart of a subject stored in the electronic memory comprises a predefined minimum value of the heart rate of the heart of a subject.

8. An insufflator as claimed in claim 6 in which a predefined maximum value of the predefined characteristic indicative of the performance of the heart of a subject is stored in the electronic memory, and on the value of the signal read by the signal processor indicative of the characteristic of the performance of the heart of the subject rising above the predefined maximum value thereof, the signal processor is programmed to operate the flow controller to reduce or pause delivery of insufflating gas to the cavity, and/or to operate the vacuum applying means to apply a vacuum to the cavity for reducing the cavity pressure.

9. An insufflator as claimed in claim 8 in which the signal processor is programmed to operate the flow controller and/or the vacuum applying means to reduce the cavity pressure until the signal read from the signal processor indicative of the characteristic of the performance of the heart of the subject commences to fall below the predefined maximum value thereof, and preferably, the predefined maximum value of the predefined characteristic indicative of the performance of the heart of a subject stored in the electronic memory comprises a predefined maximum value of the heart rate of a subject.

10. An insufflator as claimed in claim 1 in which a predefined maximum volume value is stored in the electronic memory, and the signal processor is programmed to compute the current volume of the cavity as a function of the insufflating gas delivered to the cavity, and to compare the current computed volume of the cavity with the stored predefined maximum volume value, and the signal processor is programmed to operate the flow controller to reduce or pause the flow of insufflating gas to the cavity in response to the current computed volume of the cavity exceeding the predefined maximum volume value.

11. A method for insufflating a cavity in the body of a human or animal subject, the method comprising:
delivering insufflating gas to the cavity,
monitoring insufflating of the cavity,
determining the rate of insufflating of the cavity,
comparing the determined rate of insufflating of the cavity with a stored reference value of a maximum rate of insufflating the cavity, and
controlling the rate of insufflating of the cavity to prevent the rate at which the cavity is being insufflated exceeding the stored reference value.

12. A method as claimed in claim 11 in which the rate at which the cavity is being insufflated is reduced or insufflating of the cavity is paused if the determined rate of insufflating of the cavity exceeds the stored reference value of the maximum rate of insufflating the cavity.

13. A method as claimed in claim 11 in which the reference value of the maximum rate of insufflating the cavity is stored as a function of cavity pressure, and preferably as a function of one or more of the type of cavity, the sex of the subject, the age of the subject, the weight of the subject, the height of the subject, and the body mass index of the subject, and preferably, the cavity pressure is monitored, and the rate of insufflating of the cavity is determined as a function of the cavity pressure, and advantageously, the reference value of the maximum rate of insufflating the cavity is stored as a function of the maximum increase in cavity pressure per unit time, and advantageously, the increase in the cavity pressure per unit time of the subject is compared with the reference value of the maximum increase in cavity pressure per unit time.

14. A method as claimed in claim 11 in which the reference value of the maximum rate of insufflating the cavity is stored as a function of the flow rate of the insufflating gas delivered to the cavity, and preferably, as a function of one or more of the type of cavity, the sex of the subject, the age of the subject, the weight of the subject, the height of the subject, and the body mass index of the subject, and preferably, the flow of insufflating gas to the cavity is monitored, and the rate at which the cavity is being insufflated is determined as the flow rate of insufflating gas to the cavity, and advantageously, the reference value of the maximum rate of insufflating the cavity is stored as the maximum flow rate of insufflating gas to the cavity, and preferably, the determined flow rate of insufflating gas to the cavity is compared with the reference value of the maximum flow rate of insufflating gas.

15. A method as claimed in claim 11 in which a plurality of reference values of maximum rates of insufflating the cavity are stored for respective subjects of at least one or more of different sexes, different ages, different weights, different heights, and different body mass indices, and preferably, the reference value of the maximum rate of insufflating the cavity appropriate to the cavity is determined from the stored reference values thereof from data indicative of the one or more of the sex, the age, the weight, the height, and the body mass index of the subject, and preferably, a plurality of the reference values are stored for each one of a plurality of different types of cavities for the subjects of the one or more different sexes, different ages, different weights, different heights, and different body mass indices.

16. A method as claimed in claim 11 in which a predefined minimum value of a predefined characteristic of the performance of the heart of a subject is stored, and a characteristic of the performance of the heart of the subject is monitored corresponding to the predefined characteristic indicative of the performance of the heart of a subject, the predefined minimum value of which is stored, and on the value of the monitored characteristic of the performance of the heart of the subject falling below the stored predefined minimum value thereof, the insufflating of the cavity is reduced or paused and/or a vacuum is applied to the cavity to reduce the cavity pressure.

17. A method as claimed in claim 16 in which the flow of insufflating gas to the cavity is reduced or paused and/or a vacuum is applied to the cavity until the value of the monitored characteristic of the performance of the heart of the subject commences to rise above the stored predefined minimum value thereof, and preferably, the stored predefined minimum value of the predefined characteristic indicative of the performance of the heart of a subject comprises a predefined minimum value of the heart rate of the heart of a subject.

18. A method as claimed in claim 16 in which a predefined maximum value of the predefined characteristic indicative of the performance of the heart of a subject is stored, and on the value of the monitored characteristic of the performance of the heart of the subject rising above the stored predefined maximum value thereof, the flow of insufflating gas to the cavity is reduce or paused and/or a vacuum is applied to the cavity to reduce the cavity pressure.

19. A method as claimed in claim 18 in which the flow of insufflating gas to the cavity is reduced or paused and/or a vacuum is applied to the cavity until the value of the monitored characteristic of the performance of the heart of the subject commences to fall below the stored predefined maximum value thereof, and preferably, the stored predefined maximum value of the predefined characteristic of the performance of the heart of a subject comprises a predefined maximum value of the heart rate of the heart of a subject.

20. A method as claimed in claim 11 in which the volume of the cavity is computed as a function of the insufflating gas delivered to the cavity, and if the computed volume of the cavity exceeds a predefined maximum volume value, insufflating of the cavity is reduced or paused.

21. An insufflator for insufflating a cavity in the body of a human or animal subject, the insufflator comprising:
an electronic memory storing a predefined minimum value of a predefined characteristic indicative of the performance of a heart of a subject,
a receiving means configured to receive a signal indicative of a value of a characteristic indicative of the performance of the heart of the subject, the cavity of which is being insufflated, corresponding to the predefined characteristic indicative of the performance of the heart of a subject, the predefined minimum value of which is stored in the electronic memory, and
a signal processor programmed to read the signal indicative of the value of the characteristic indicative of the performance of the heart of the subject from the receiving means, and to reduce or pause insufflating of the cavity of the subject, and/or to evacuate the cavity of the subject to reduce the cavity pressure in response to the value of the signal indicative of the characteristic of the performance of the heart of the subject falling below the predefined minimum value thereof.

22. An insufflator as claimed in claim 21 in which the signal processor is programmed to reduce the cavity pressure until the value of the signal read from the receiving means by the signal processor indicative of the characteristic of the performance of the heart of the subject commences to rise above the stored predefined minimum value thereof, and preferably, the predefined minimum value of the predefined characteristic indicative of the performance of the heart of a subject stored in the electronic memory comprises a predefined minimum value of the heart rate of a subject.

23. An insufflator as claimed in claim 21 in which a predefined maximum value of the predefined characteristic indicative of the performance of the heart of a subject is stored in the electronic memory, and the signal processor is programmed in response to the value of the signal read from the receiving means indicative of the characteristic of the performance of the heart of the subject rising above the predefined maximum value thereof to reduce or pause insufflating of the cavity of the subject, and/or to evacuate the cavity of the subject to reduce the cavity pressure.

24. An insufflator as claimed in claim 23 in which the signal processor is programmed to reduce the cavity pressure until the value of the signal read from the receiving means by the signal processor indicative of the characteristic of the performance of the heart of the subject commences to fall below the predefined maximum value.

25. A method for insufflating a cavity in the body of a human or animal subject, the method comprising:
storing a predefined minimum value of a predefined characteristic indicative of the performance of a heart of a subject,
monitoring a value of a characteristic indicative of the performance of the heart of the subject, the cavity of which is being insufflated corresponding to the predefined characteristic indicative of the performance of the heart of a subject, the predefined minimum value of which is stored, and
on the value of the signal indicative of the characteristic of the performance of the heart of the subject falling below the predefined minimum value thereof reducing or pausing insufflating of the cavity of the subject, and/or evacuating the cavity of the subject to reduce the cavity pressure.

26. A method as claimed in claim 25 in which the cavity pressure is reduced until the value of the signal read by the signal processor indicative of the characteristic of the performance of the heart of the subject commences to rise above the stored predefined minimum value thereof, and preferably, the predefined minimum value of the predefined characteristic indicative of the performance of the heart of a subject is stored as a predefined minimum value of the heart rate of a subject.

27. A method as claimed in claim 25 in which a predefined maximum value of the predefined characteristic indicative of the performance of the heart of a subject is stored, and on the value of the received signal indicative of the characteristic of the performance of the heart of the subject rising above the predefined maximum value thereof insufflating of the cavity of the subject is reduced or paused, and/or the cavity of the subject is evacuated to depressurise the cavity.

28. A method as claimed in claim 27 in which the cavity pressure is reduced until the value of the signal indicative of the characteristic of the performance of the heart of the subject commences to fall below the maximum predefined value thereof.

29. An insufflator for insufflating a cavity in the body of a human or animal subject, the insufflator comprising:
a flow controller for controlling delivery of insufflating gas to the cavity,
an electronic memory for storing a predefined maximum volume value for a cavity,
a flow sensor for monitoring flow of insufflating gas to the cavity and for producing a signal indicative of the flow of insufflating gas to the cavity, and
a signal processor, the signal processer being programmed
to read the signal from the flow sensor indicative of the flow of insufflating gas to the cavity,
to determine the volume of the cavity from the signal read from the flow sensor,
to compare the determined volume of the cavity with the stored predefined maximum volume value for the cavity, and
to control the flow controller to reduce or pause insufflating of the cavity in response to the computed volume of the cavity exceeding the predefined maximum volume value.

30. A method for insufflating a cavity in the body of a human or animal subject, the method comprising:
delivering insufflating gas to the cavity,
monitoring the flow of insufflating gas to the cavity,
determining the volume of the cavity as a function of the flow of insufflating gas to the cavity,
comparing the determined volume of the cavity with a stored predefined maximum volume value, and
controlling insufflating of the cavity to reduce the volume of the cavity if the determined volume of the cavity exceeds the predefined maximum volume value.
